SEQUENCE LISTING (181 sequences)

SEQ ID NO: 1 - protein, 28 aa, Artificial Sequence
  Artifically synthesized peptide
RVIEVVQGACRAIRHIPRRIRQGLERIL

SEQ ID NO: 2 - protein, 21 aa, Artificial Sequence
  Artifically Synthesized peptide
YHRLRDLLLIVTRIVELLGRR

SEQ ID NO: 3 - protein, 28 aa, Artificial Sequence
  Artificially synthesized peptide
DLWETLRRGGRWILAIPRRIREGLELTL

SEQ ID NO: 4 - protein, 25 aa, Artificial Sequence
  Artificially synthesized peptide
FLIRQLIRLLTWLFSNCRTLLSRVY

SEQ ID NO: 5 - protein, 28 aa, Artificial Sequence
  Artificially synthesized peptide
LLSRVYQILQPILQRLSATLQRIREVLR

SEQ ID NO: 6 - protein, 29 aa, Artificial Sequence
  Artificially synthesized peptide
RIAGYGLRGLAVIIRICIRGLNLIFEIIR

SEQ ID NO: 7 - protein, 21 aa, Artificial Sequence
  Artificially synthesized peptide
RVIEVVQGACRAIRHIPRRIR

SEQ ID NO: 8 - protein, 17 aa, Artificial Sequence
  Artificially synthesized peptide
VVQGACRAIRHIPRRIR

SEQ ID NO: 9 - protein, 14 aa, Artificial Sequence
  Artificially synthesized peptide
GACRAIRHIPRRIR

SEQ ID NO: 10 - protein, 21 aa, Artificial Sequence
  Artificially synthesized peptide
RVIRVVQGACRAIRHIPRRIR

SEQ ID NO: 11 - protein, 21 aa, Artificial Sequence
  Artificially synthesized peptide
RVIEVVRGACRAIRHIPRRIR

SEQ ID NO: 12 - protein, 21 aa, Artificial Sequence
  Artificially synthesized peptide
RVIEVVQGICRAIRHIPRRIR

SEQ ID NO: 13 - protein, 21 aa, Artificial Sequence
  Artificially synthesized peptide
RVIAVVQGACRAIRRIPRRIR

SEQ ID NO: 14 - protein, 28 aa, Artificial Sequence
  Artificially synthesized peptide
RVIRVVQGACRAIRHIPRRIRQGLERIL

SEQ ID NO: 15 - protein, 28 aa, Artificial Sequence
  Artificially synthesized peptide
RVIEVVRGACRAIRHIPRRIRQGLERIL

SEQ ID NO: 16 - protein, 28 aa, Artificial Sequence
  Artificially synthesized peptide
RVIEVVQGICRAIRHIPRRIRQGLERIL

SEQ ID NO: 17 - protein, 28 aa, Artificial Sequence
  Artificially synthesized peptide
RVISVVQGACRAIRRIPRRIRQGLERIL

SEQ ID NO: 18 - protein, 28 aa, Artificial Sequence
  Artificially synthesized peptide
RVIEVVQGACRAIRHIPRRIRQILERIL

SEQ ID NO: 19 - protein, 28 aa, Artificial Sequence
  Artificially synthesized peptide
RVIEVVQGACRAIRHIPRRIRQGLRRIL

SEQ ID NO: 20 - protein, 21 aa, Artificial Sequence
  Artificially synthesized peptide
RVIRVVQGACRAIRHIPRRIR

SEQ ID NO: 21 - protein, 21 aa, Artificial Sequence
  Artificially synthesized peptide
RVIEVVRGACRAIRHIPRRIR

SEQ ID NO: 22 - protein, 21 aa, Artificial Sequence
  Artificially synthesized peptide
RVIEVVQGICRAIRHIPRRIR

SEQ ID NO: 23 - protein, 21 aa, Artificial Sequence
  Artificially synthesized peptide
RVIEVVQGACRRIRRIPRRIR

SEQ ID NO: 24 - protein, 28 aa, Artificial Sequence
  Artificially synthesized peptide
RVIRVVQGACRAIRHIPRRIRQGLERIL

SEQ ID NO: 25 - protein, 28 aa, Artificial Sequence
  Artificially synthesized peptide
RVIEVVRGACRAIRHIPRRIRQGLERIL

SEQ ID NO: 26 - protein, 28 aa, Artificial Sequence
  Artificially synthesized peptide
RVIEVVQGICRAIRHIPRRIRQGLERIL

SEQ ID NO: 27 - protein, 28 aa, Artificial Sequence
  Artificially synthesized peptide
RVIEVVQGACRAIRRIPRRIRQGLERIL

SEQ ID NO: 28 - protein, 17 aa, Artificial Sequence
  Artificially synthesized peptide
VVRGACRAIRHIPRRIR

SEQ ID NO: 29 - protein, 17 aa, Artificial Sequence
  Artificially synthesized peptide
VVQGICRAIRHIPRRIR

SEQ ID NO: 30 - protein, 17 aa, Artificial Sequence
  Artificially synthesized peptide
VVQRACRAIRRIPRRIR

SEQ ID NO: 31 - protein, 23 aa, Artificial Sequence
  Artificially synthesized peptide
VVRGACRAIRHIPRRIRGLERIL

SEQ ID NO: 32 - protein, 23 aa, Artificial Sequence
  Artificially synthesized peptide
VVQGICRAIRHIPRRIRGLERIL

SEQ ID NO: 33 - protein, 23 aa, Artificial Sequence
  Artificially synthesized peptide
VVQGACRAIRRIPRRIRGLERIL

SEQ ID NO: 34 - protein, 14 aa, Artificial Sequence
  Artificially synthesized peptide
GACRAIRRIPRRIR

SEQ ID NO: 35 - protein, 20 aa, Artificial Sequence
  Artificially synthesized peptide
GACRAIRRIPRRIRGLERIL

SEQ ID NO: 36 - protein, 17 aa, Artificial Sequence
  Artificially synthesized peptide
VVQRACRAIRHIPRRIR

SEQ ID NO: 37 - protein, 14 aa, Artificial Sequence
  Artificially synthesized peptide
RACRAIRHIPRRIR

SEQ ID NO: 38 - protein, 21 aa, Artificial Sequence
  Artificially synthesized peptide
RVIRVVRGACRAIRHIPRRIR

SEQ ID NO: 39 - protein, 21 aa, Artificial Sequence
  Artificially synthesized peptide
RVIRVVRGACRAIRHIPRRIR

SEQ ID NO: 40 - protein, 17 aa, Artificial Sequence
  Artificially synthesized peptide
RRIRHIPRAIRVVQGAC

SEQ ID NO: 41 - protein, 21 aa, Artificial Sequence
  Artificially synthesized peptide
RIRRPIHRIARCAGQVVEIVR

SEQ ID NO: 42 - protein, 28 aa, Artificial Sequence
  Artificially synthesized peptide
LIRELGQRIRRPIHRIARCAGQVVEIVR

SEQ ID NO: 43 - protein, 28 aa, Artificial Sequence
  Artificially synthesized peptide
LIRELGQRIRRPIHRIARCAGQVVRIVR

SEQ ID NO: 44 - protein, 28 aa, Artificial Sequence
  Artificially synthesized peptide
LIRELGQRIRRPIHRIARCAGRVVEIVR

SEQ ID NO: 45 - protein, 28 aa, Artificial Sequence
  Artificially synthesized peptide
LIRELGQRIRRPIHRIARCIGQVVEIVR

SEQ ID NO: 46 - protein, 28 aa, Artificial Sequence
  Artificially synthesized peptide
LIRELGQRIRRPIRRIARCAGQVVEIVR

SEQ ID NO: 47 - protein, 28 aa, Artificial Sequence
  Artificially synthesized peptide
LIRRLGQRIRRPIHRIARCAGQVVEIVR

SEQ ID NO: 48 - protein, 28 aa, Artificial Sequence
  Artificially synthesized peptide
LIRRLGQRIRRPIHRIARCAGQVVEIVR

SEQ ID NO: 49 - protein, 21 aa, Artificial Sequence
  Artificially synthesized peptide
RIRRPIHRIARCAGQVVEIVR

SEQ ID NO: 50 - protein, 21 aa, Artificial Sequence
  Artificially synthesized peptide
RIRRPIHRIARCAGQVVRIVR

SEQ ID NO: 51 - protein, 21 aa, Artificial Sequence
  Artificially synthesized peptide
RIRRPIHRIARCAGRVVEIVR

SEQ ID NO: 52 - protein, 21 aa, Artificial Sequence
  Artificially synthesized peptide
RIRRPIHRIARCIGQVVEIVR

SEQ ID NO: 53 - protein, 21 aa, Artificial Sequence
  Artificially synthesized peptide
RIRRPIRRIARCAGQVVEIVR

SEQ ID NO: 54 - protein, 21 aa, Artificial Sequence
  Artificially synthesized peptide
RIRRPIHRIIRCIGQVVRIVR

SEQ ID NO: 55 - protein, 21 aa, Artificial Sequence
  Artificially synthesized peptide
RIRRPIRRIIRCIGQVVEIVR

SEQ ID NO: 56 - protein, 24 aa, Artificial Sequence
  Artificially synthesized peptide
LIRELGQRIRRPIHRIARCAGQVV

SEQ ID NO: 57 - protein, 24 aa, Artificial Sequence
  Artificially synthesized peptide
LIRELRQRIRRPIHRIARCARQVV

SEQ ID NO: 58 - protein, 24 aa, Artificial Sequence
  Artificially synthesized peptide
LIRELGQRIRRPIHRIARCAGRVV

SEQ ID NO: 59 - protein, 24 aa, Artificial Sequence
  Artificially synthesized peptide
LIRELGQRIRRPIHRIARCIGQVV

SEQ ID NO: 60 - protein, 24 aa, Artificial Sequence
  Artificially synthesized peptide
LIRELGQRIRRPIRRIARCAGQVV

SEQ ID NO: 61 - protein, 24 aa, Artificial Sequence
  Artificially synthesized peptide
LIRELGIRIRRPIHRIARCAGQVV

SEQ ID NO: 62 - protein, 24 aa, Artificial Sequence
  Artificially synthesized peptide
LIRRLGQRIRRPIHRIARCAGQVV

SEQ ID NO: 63 - protein, 21 aa, Artificial Sequence
  Artificially synthesized peptide
LIRELGQRIRRPIHRIARCAG

SEQ ID NO: 64 - protein, 21 aa, Artificial Sequence
  Artificially synthesized peptide
LIRELGQRIRRPIHRIARCAR

SEQ ID NO: 65 - protein, 21 aa, Artificial Sequence
  Artificially synthesized peptide
LIRELGQRIRRPIHRIARCAI

SEQ ID NO: 66 - protein, 21 aa, Artificial Sequence
  Artificially synthesized peptide
LIRELGQRIRRPIHRIARCIG

SEQ ID NO: 67 - protein, 21 aa, Artificial Sequence
  Artificially synthesized peptide
LIRELGQRIRRPIRRIARCAG

SEQ ID NO: 68 - protein, 21 aa, Artificial Sequence
  Artificially synthesized peptide
LIRELGIRIRRPIHRIARCAG

SEQ ID NO: 69 - protein, 21 aa, Artificial Sequence
  Artificially synthesized peptide
LIRRLGQRIRRPIHRIARCAG

SEQ ID NO: 70 - protein, 15 aa, Artificial Sequence
  Artificially synthesized peptide
RAIRRAIRGAPRAIL

SEQ ID NO: 71 - protein, 19 aa, Artificial Sequence
  Artificially synthesized peptide
RAIRRAIRGAPRAILRAIL

SEQ ID NO: 72 - protein, 28 aa, Artificial Sequence
  Artificially synthesized peptide
KVIEVVQGACKAIKHIPKKIKQGLEKIL

SEQ ID NO: 73 - protein, 20 aa, Artificial Sequence
  Artificially synthesized peptide
LWETLRRGGRWILAIPRRIR

SEQ ID NO: 74 - protein, 28 aa, Artificial Sequence
  Artificially synthesized peptide
DLWETLRRIIRWILAIPRRIRQGLELTL

SEQ ID NO: 75 - protein, 28 aa, Artificial Sequence
  Artificially synthesized peptide
DLWETLRRGGRWILAIPRRIRQGLELCL

SEQ ID NO: 76 - protein, 28 aa, Artificial Sequence
  Artificially synthesized peptide
DLWETLRRGCRWILAIPRRIRQGLELTL

SEQ ID NO: 77 - protein, 28 aa, Artificial Sequence
  Artificially synthesized peptide
DLWETLRRIIRWILAIPRRIRQGLELCL

SEQ ID NO: 78 - protein, 27 aa, Artificial Sequence
  Artificially synthesized peptide
LWETLRRGGRWILAIPRRIRQGLELTL

SEQ ID NO: 79 - protein, 27 aa, Artificial Sequence
  Artificially synthesized peptide
LWETLRRGGRWILAIPRRIRQGLELCL

SEQ ID NO: 80 - protein, 27 aa, Artificial Sequence
  Artificially synthesized peptide
LWETLRRGCRWILAIPRRIRQGLELTL

SEQ ID NO: 81 - protein, 27 aa, Artificial Sequence
  Artificially synthesized peptide
LWRTLRRGGRWILAIPRRIRQGLELTL

SEQ ID NO: 82 - protein, 27 aa, Artificial Sequence
  Artificially synthesized peptide
LWETLRRGGRWILAIPRRIRQGLRLTL

SEQ ID NO: 83 - protein, 27 aa, Artificial Sequence
  Artificially synthesized peptide
LWETLRRGGRWILAIPRRIRRGLELTL

SEQ ID NO: 84 - protein, 27 aa, Artificial Sequence
  Artificially synthesized peptide
LWETLRRGGRWILAIPRRIRRQIELTL

SEQ ID NO: 85 - protein, 27 aa, Artificial Sequence
  Artificially synthesized peptide
LWELLRRGGRWILAIPRRIRQGLELTL

SEQ ID NO: 86 - protein, 27 aa, Artificial Sequence
  Artificially synthesized peptide
LWRLLRRGGRWILAIPRRIRQGLELTL

SEQ ID NO: 87 - protein, 21 aa, Artificial Sequence
  Artificially synthesized peptide
DLWETLRRIIRWILAIPRRIR

SEQ ID NO: 88 - protein, 21 aa, Artificial Sequence
  Artificially synthesized peptide
DLWETLRRGGRWILAIPRRIR

SEQ ID NO: 89 - protein, 21 aa, Artificial Sequence
  Artificially synthesized peptide
DLWETLRRGCRWILAIPRRIR

SEQ ID NO: 90 - protein, 20 aa, Artificial Sequence
  Artificially synthesized peptide
LWETLRRGGRWILAIPRRIR

SEQ ID NO: 91 - protein, 20 aa, Artificial Sequence
  Artificially synthesized peptide
LWETLRRIIRWILAIPRRIR

SEQ ID NO: 92 - protein, 20 aa, Artificial Sequence
  Artificially synthesized peptide
LWETLRRGCRWILAIPRRIR

SEQ ID NO: 93 - protein, 20 aa, Artificial Sequence
  Artificially synthesized peptide
LWETLRRGGRWILAIPRRIR

SEQ ID NO: 94 - protein, 20 aa, Artificial Sequence
  Artificially synthesized peptide
LWETLRRGCRWILAIPRRIR

SEQ ID NO: 95 - protein, 20 aa, Artificial Sequence
  Artificially synthesized peptide
LWETLRRIIRWILAIPRRIR

SEQ ID NO: 96 - protein, 20 aa, Artificial Sequence
  Artificially synthesized peptide
LWELLRRGGRWILAIPRRIR

SEQ ID NO: 97 - protein, 20 aa, Artificial Sequence
  Artificially synthesized peptide
LWRLLRRGGRWILAIPRRIR

SEQ ID NO: 98 - protein, 16 aa, Artificial Sequence
  Artificially synthesized peptide
LRRGGRWILAIPRRIR

SEQ ID NO: 99 - protein, 20 aa, Artificial Sequence
  Artificially synthesized peptide
LWETLRRGGRWILAIPRRIL

SEQ ID NO: 100 - protein, 16 aa, Artificial Sequence
  Artificially synthesized peptide
LRRGGRWILAIPRAIL

SEQ ID NO: 101 - protein, 20 aa, Artificial Sequence
  Artificially synthesized peptide
LWETLRRGGRWILAIPREIL

SEQ ID NO: 102 - protein, 16 aa, Artificial Sequence
  Artificially synthesized peptide
LRRGGRWILAIPREIL

SEQ ID NO: 103 - protein, 18 aa, Artificial Sequence
  Artificially synthesized peptide
WILAIPRRIRGGRLWETL

SEQ ID NO: 104 - protein, 18 aa, Artificial Sequence
  Artificially synthesized peptide
WETLPRRIRGGRLWILAI

SEQ ID NO: 105 - protein, 20 aa, Artificial Sequence
  Artificially synthesized peptide
RIRRPIALIWRGGRRLTEWL

SEQ ID NO: 106 - protein, 28 aa, Artificial Sequence
  Artificially synthesized peptide
DLWETLKKGGRWILAIPRRIKQGLELTL

SEQ ID NO: 107 - protein, 27 aa, Artificial Sequence
  Artificially synthesized peptide
LWETLGRVGRWVLAIPRRIRQGLELAL

SEQ ID NO: 108 - protein, 21 aa, Artificial Sequence
  Artificially synthesized peptide
YHRLRRLLLIVTRIVELLGRR

SEQ ID NO: 109 - protein, 21 aa, Artificial Sequence
  Artificially synthesized peptide
YHRLRDLLRIVTRIVELLGRR

SEQ ID NO: 110 - protein, 21 aa, Artificial Sequence
  Artificially synthesized peptide
YHRLRDLLLIVRRIVELLGRR

SEQ ID NO: 111 - protein, 22 aa, Artificial Sequence
  Artificially synthesized peptide
YHRLRDLLLIIVTRIVRLLGRR

SEQ ID NO: 112 - protein, 21 aa, Artificial Sequence
  Artificially synthesized peptide
YHRLRDLLLIVTRIVCLLGRR

SEQ ID NO: 113 - protein, 21 aa, Artificial Sequence
  Artificially synthesized peptide
YHRLRDLLLIVRRIVCLLGRR

SEQ ID NO: 114 - protein, 21 aa, Artificial Sequence
  Artificially synthesized peptide
YHRRLRDLLIVTRIVELLGRR

SEQ ID NO: 115 - protein, 18 aa, Artificial Sequence
  Artificially synthesized peptide
YHRLRRLLLIVTRIVELL

SEQ ID NO: 116 - protein, 18 aa, Artificial Sequence
  Artificially synthesized peptide
YHRLRDLLRIVTRIVELL

SEQ ID NO: 117 - protein, 18 aa, Artificial Sequence
  Artificially synthesized peptide
YHRLRDLLLIVRRIVELL

SEQ ID NO: 118 - protein, 18 aa, Artificial Sequence
  Artificially synthesized peptide
YHRLRDLLLIVTRIVRLL

SEQ ID NO: 119 - protein, 18 aa, Artificial Sequence
  Artificially synthesized peptide
YHRLRDLLLIVTRIVCLL

SEQ ID NO: 120 - protein, 18 aa, Artificial Sequence
  Artificially synthesized peptide
YHRLRDLLLIVRRIVCLL

SEQ ID NO: 121 - protein, 18 aa, Artificial Sequence
  Artificially synthesized peptide
YHRLLRDLLIVTRIVELL

SEQ ID NO: 122 - protein, 18 aa, Artificial Sequence
  Artificially synthesized peptide
YHRLRDLLLIVTRIVELL

SEQ ID NO: 123 - protein, 21 aa, Artificial Sequence
  Artificially synthesized peptide
RRGLLEVIRTVILPRRLLDRL

SEQ ID NO: 124 - protein, 18 aa, Artificial Sequence
  Artificially synthesized peptide
YHRLRDLALIVTRIVELL

SEQ ID NO: 125 - protein, 18 aa, Artificial Sequence
  Artificially synthesized peptide
RRGLLRVIRTVILALDIL

SEQ ID NO: 126 - protein, 21 aa, Artificial Sequence
  Artificially synthesized peptide
RRGLLEVIRTVILLLDRLRHY

SEQ ID NO: 127 - protein, 21 aa, Artificial Sequence
  Artificially synthesized peptide
RRGLLEVIRTVILALDRLRHY

SEQ ID NO: 128 - protein, 18 aa, Artificial Sequence
  Artificially synthesized peptide
RRGLLEVIRTVILALDRL

SEQ ID NO: 129 - protein, 18 aa, Artificial Sequence
  Artificially synthesized peptide
YHRLRDLLLIVCRIVELL

SEQ ID NO: 130 - protein, 21 aa, Artificial Sequence
  Artificially synthesized peptide
YHRLRDLLLIVCRIVELLGRR

SEQ ID NO: 131 - protein, 21 aa, Artificial Sequence
  Artificially synthesized peptide
YHRLRDLLLIVTRIVELLGRR

SEQ ID NO: 132 - protein, 18 aa, Artificial Sequence
  Artificially synthesized peptide
RRGLLEVIRCVILLLDRL

SEQ ID NO: 133 - protein, 18 aa, Artificial Sequence
  Artificially synthesized peptide
RRGLLRVIRTVILLLDRL

SEQ ID NO: 134 - protein, 18 aa, Artificial Sequence
  Artificially synthesized peptide
RRGLLEVIRTVILLLRRL

SEQ ID NO: 135 - protein, 21 aa, Artificial Sequence
  Artificially synthesized peptide
RRGLLEVIRCVILLLDRLRHY

SEQ ID NO: 136 - protein, 21 aa, Artificial Sequence
  Artificially synthesized peptide
RRGLLRVIRTVILLLDRLRHY

SEQ ID NO: 137 - protein, 21 aa, Artificial Sequence
  Artificially synthesized peptide
RRGLLEVIRTVILLLRRLRHY

SEQ ID NO: 138 - protein, 20 aa, Artificial Sequence
  Artificially synthesized peptide
YHKLKLLLIVTKIVELLGKK

SEQ ID NO: 139 - protein, 22 aa, Artificial Sequence
  Artificially synthesized peptide
FLIRQLIRQLLTWQPILQYILQ

SEQ ID NO: 140 - protein, 25 aa, Artificial Sequence
  Artificially synthesized peptide
FLIRQLIRLLTWLFSNCRTLLSEVY

SEQ ID NO: 141 - protein, 21 aa, Artificial Sequence
  Artificially synthesized peptide
FLIRQLIRLLTWLFSNCRTLL

SEQ ID NO: 142 - protein, 21 aa, Artificial Sequence
  Artificially synthesized peptide
LLTRCNSFLWTLLRILQRILF

SEQ ID NO: 143 - protein, 25 aa, Artificial Sequence
  Artificially synthesized peptide
FLIRQLIRLLTWLFPNCRTLLSRVY

SEQ ID NO: 144 - protein, 25 aa, Artificial Sequence
  Artificially synthesized peptide
YVRSLLTRCNSFLWTLLRILQRILF

SEQ ID NO: 145 - protein, 25 aa, Artificial Sequence
  Artificially synthesized peptide
FLIKQLIKLLTWLFSNCLTLLSKVY

SEQ ID NO: 146 - protein, 23 aa, Artificial Sequence
  Artificially synthesized peptide
RLVERIRQLTASRQLIPQLIQYV

SEQ ID NO: 147 - protein, 23 aa, Artificial Sequence
  Artificially synthesized peptide
RLVRRIRQLTASRQLIPQLIQYV

SEQ ID NO: 148 - protein, 27 aa, Artificial Sequence
  Artificially synthesized peptide
LLSRVYQILQPILQRLSATLQAIREVL

SEQ ID NO: 149 - protein, 28 aa, Artificial Sequence
  Artificially synthesized peptide
LLSRVYQILQPILQRLCATLQRIREVLR

SEQ ID NO: 150 - protein, 28 aa, Artificial Sequence
  Artificially synthesized peptide
RLVERIRQLTASLRQLIPQLIQYVRSLL

SEQ ID NO: 151 - protein, 28 aa, Artificial Sequence
  Artificially synthesized peptide
LLSKVYQILQPILQKLSATLQKIKEVLK

SEQ ID NO: 152 - protein, 25 aa, Artificial Sequence
  Artificially synthesized peptide
RLLTWLFSNCRTLLSRVYQILQPIL

SEQ ID NO: 153 - protein, 25 aa, Artificial Sequence
  Artificially synthesized peptide
RLLTWLFSNRRTLLSRVYQILQEIL

SEQ ID NO: 154 - protein, 22 aa, Artificial Sequence
  Artificially synthesized peptide
RLLTWLRRTLLSRVYQILQEIL

SEQ ID NO: 155 - protein, 29 aa, Artificial Sequence
  Artificially synthesized peptide
RIAGYGLRGLAVIIRCIIRGLNLIFEIIR

SEQ ID NO: 156 - protein, 29 aa, Artificial Sequence
  Artificially synthesized peptide
RIAGYGLRGLAVIIRIICRGLNLIFEIIR

SEQ ID NO: 157 - protein, 30 aa, Artificial Sequence
  Artificially synthesized peptide
RIAGYGLRGLAVIPRRICIRGLNLIFEIIR

SEQ ID NO: 158 - protein, 29 aa, Artificial Sequence
  Artificially synthesized peptide
RIIEFILNLGRICIRIIVALGRLGYGAIR

SEQ ID NO: 159 - protein, 29 aa, Artificial Sequence
  Artificially synthesized peptide
KIAGYGLKGLAVIIKICIKGLNLIFEIIK

SEQ ID NO: 160 - protein, 28 aa, Artificial Sequence
  Artificially synthesized peptide
RVIRVVQGACRAIRHIPRRIRQGLRRIL

SEQ ID NO: 161 - protein, 28 aa, Artificial Sequence
  Artificially synthesized peptide
RVIEVVQGACRAIEHIPRRIEQGLERIL

SEQ ID NO: 162 - protein, 28 aa, Artificial Sequence
  Artificially synthesized peptide
RVIEVVQGACRAIEHIPRRIRQGLERIL

SEQ ID NO: 163 - protein, 28 aa, Artificial Sequence
  Artificially synthesized peptide
RVIEVVQGACRAIRHIPRRIEQGLERIL

SEQ ID NO: 164 - protein, 28 aa, Artificial Sequence
  Artificially synthesized peptide
RVIEVVQGACRASRHIPRRIRQGLERIL

SEQ ID NO: 165 - protein, 28 aa, Artificial Sequence
  Artificially synthesized peptide
RVIEVVQGACRAIRHIPRRSRQGLERIL

SEQ ID NO: 166 - protein, 25 aa, Artificial Sequence
  Artificially synthesized peptide
FLIRQLIELLTWLFSNCRTLLSEVY

SEQ ID NO: 167 - protein, 28 aa, Artificial Sequence
  Artificially synthesized peptide
LLSEVYQILQPILQELSATLQRIREVLR

SEQ ID NO: 168 - protein, 21 aa, Artificial Sequence
  Artificially synthesized peptide
YHELRDLLLIVTRIVELLGRE

SEQ ID NO: 169 - protein, 28 aa, Artificial Sequence
  Artificially synthesized peptide
RVIEVVQGAYRAIRHIPRRIRQGLERIL

SEQ ID NO: 170 - protein, 28 aa, Artificial Sequence
  Artificial peptide derived from HIV-1
RVIRVVQRACRAIRHIVRRIRQGLRRIL

SEQ ID NO: 171 - protein, 31 aa, Artificial Sequence
  Artificial peptide derived from HIV-1
RVIRVVQRACRAIRHIVRRIRQGLRRILRVV

SEQ ID NO: 172 - protein, 31 aa, Artificial Sequence
  Artificial peptide derived from HIV-1
RWIRVVQRWCRAIRHIWRRIRQGLRRWLRVV

SEQ ID NO: 173 - protein, 12 aa, Artificial Sequence
  Artificial peptide derived from HIV-1
RVVRVVRRVVRR

SEQ ID NO: 174 - protein, 24 aa, Artificial Sequence
  Artificial peptide derived from HIV-1
RRVVRRVRRVVRRVVRVVRRVVRR

SEQ ID NO: 175 - protein, 36 aa, Artificial Sequence
  Artificial peptide derived from HIV-1
VRRVVRRVVRVVRRVVRRVRRVVRRVVRVVRRVVRR

SEQ ID NO: 176 - protein, 42 aa, Artificial Sequence
  Artificial peptide derived from HIV-1
RRVVRRVRRVVRRVVRVVRRVVRRVRRVVRRVVRVVRRVVRR

SEQ ID NO: 177 - protein, 48 aa, Artificial Sequence
  Artificial peptide derived from HIV-1
RVVRVVRRVVRRVRRVVRRVVRVVRRVVRRVRRVVRRVVRVVRRVVRR

SEQ ID NO: 178 - protein, 12 aa, Artificial Sequence
  Artificial peptide derived from HIV-1
RVVRVVRRWVRR

SEQ ID NO: 179 - protein, 24 aa, Artificial Sequence
  Artificial peptide derived from HIV-1
RRWVRRVRRVWRRVVRVVRRWVRR

SEQ ID NO: 180 - protein, 36 aa, Artificial Sequence
  Artificial peptide derived from HIV-1
VRRVWRRVVRVVRRWVRRVRRVWRRVVRVVRRWVRR

SEQ ID NO: 181 - protein, 48 aa, Artificial Sequence
  Artificial peptide derived from HIV-1
RVVRVVRRWVRRVRRVWRRVVRVVRRWVRRVRRVWRRVVRVVRRWRVV

(19) United States
(12) Patent Application Publication
(10) Pub. No.: US 2010/0150985 A1
(43) Pub. Date: Jun. 17, 2010

(54) Dental Implant, Endodontic Instrument, and Dental Filling Material Coated with a Peptide-Based Antimicrobial and Methods of Using and Making the Same

(76) Inventors: George Just, Monroeville, PA (US); Kenneth J. Polk, Pittsburgh, PA (US)

Correspondence Address: BLYNN L. SHIDELER;THE BLK LAW GROUP, 3500 BROKKTREE ROAD, SUITE 200, WEXFORD, PA, 15090, US

(21) Appl. No.: 12/109,105

(22) Filed: Apr. 24, 2008

(51) Int. Cl. A61C 8/00; A61K 38/02; B05D 3/00; A61K 38/16; A61K 38/10; A61P 31/00; A61C 5/04

(52) U.S. Cl. 424/435; 514/2; 427/2.26; 514/12; 514/13; 514/14; 433/81

(57) ABSTRACT

Dental implants and endodontic instruments are coated with an antimicrobial peptide-based coating. Methods of coating the dental implants and endodontic instruments with the antimicrobial peptide-based coating are disclosed together with treating a subject with the coated dental implant and endodontic instruments to prevent or lessen bacterial infections in the subject.

### CROSS-REFERENCE TO RELATED APPLICATIONS

TO BE SUPPLIED

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

### BACKGROUND

1. Field of Invention

The present invention is directed towards dental implants, endodontic instruments, and dental filling materials, in particular such apparatus coated with a peptide based antimicrobial and methods of using and making the same.

2. Description of Related Art

Devices for use in and on the human body are well known. The chemical composition of the surfaces of such devices plays a pivotal role in dictating the overall efficacy of the devices. Additionally, it is known that providing such devices with an antimicrobial surface is advantageous.

A wide variety of bactericidal and bacteriostatic coatings have been developed. See also, for example, U.S. Patent Application Publication 2008-0063688 for the purpose of the background information described therein, wherein this publication is incorporated herein by reference.

Additionally in the dental fields please see the following:
1 U.S. Pat. No. 6,553,996 Dental appliance with antimicrobial additive
2 U.S. Pat. No. 6,267,590 Antimicrobial dental products
3 U.S. Pat. No. 6,071,528 Adhesive antimicrobial and/or reparative dentin stimulating dental compositions and methods for forming and using such compositions
4 U.S. Pat. No. 5,770,182 Methods for treating teeth with anticariogenic and antimicrobial dental compositions
5 U.S. Pat. No. 5,733,949 Antimicrobial adhesive composition for dental uses
6 U.S. Pat. No. 5,560,906 Non-alcoholic antimicrobial mouthwash for removal of dental plaque
7 U.S. Pat. No. 4,883,534 Benzoin antimicrobial dental varnishes
8 U.S. Pat. No. 4,496,322 Benzoin antimicrobial dental varnishes

These patents are incorporated herein by reference.

U.S. Patent Application Publication 2002-0169279 is directed to peptides having antimicrobial activity (antimicrobial peptides). The antimicrobial peptides of this disclosure are analogs of the Lentivirus Lytic Peptide 1 (LLP1) amino acid sequence. This disclosure further teaches peptides referred to as the Lytic Base Unit (LBU) peptides derived from the LLP1 analogs, also having antimicrobial activity. See also U.S. Patent Application Publications 20020188102, and 20030036627. These publications are incorporated herein by reference.

U.S. Patent Application Publication 2007-0244044 discloses a peptide comprising amino acids including an amino acid selected from the group consisting of hydrophobic amino acids and/or cationic amino acids, for use as a medicaments. This publication is incorporated herein by reference.

Biomedical devices with antimicrobial coatings are discussed in U.S. Patent Application Publication 2004-0126409, wherein one or more surfaces of the device are coated with a cationic peptide, cationic proteins, or mixtures thereof to impart antimicrobial properties to the surface, which is incorporated herein by reference.

For further background on antimicrobial peptides please see:
1 U.S. Pat. No. 7,348,402 Antimicrobial peptide, its analogs and antimicrobial composition comprising them
2 U.S. Pat. No. 7,271,239 D-isomers of antimicrobial peptide
3 U.S. Pat. No. 7,223,840 Antimicrobial peptide
4 U.S. Pat. No. 7,176,276 Antimicrobial peptide and use thereof
5 U.S. Pat. No. 7,041,647 Synthetic peptide having an ionophoric and antimicrobial activity
6 U.S. Pat. No. 6,809,181 Human beta-defensin-3 (HBD-3), a highly cationic beta-defensin antimicrobial peptide
7 U.S. Pat. No. 6,699,689 Mass production method of antimicrobial peptide and DNA construct and expression system thereof
8 U.S. Pat. No. 6,420,116 Antimicrobial peptide
9 U.S. Pat. No. 6,358,921 Antimicrobial peptide compositions and method
10 U.S. Pat. No. 6,316,594 Antimicrobial peptide isolated from parasilurus asotus and its uses
11 U.S. Pat. No. 6,235,973 Expression of magainin and PGL classes of antimicrobial peptide genes in plants, and their use in creating resistance to multiple plant pathogens
12 U.S. Pat. No. 6,183,992 Method for mass production of antimicrobial peptide
13 U.S. Pat. No. 6,172,185 Antimicrobial cationic peptide derivatives of bactenecin
14 U.S. Pat. No. 6,143,498 Antimicrobial peptide
15 U.S. Pat. No. 6,042,848 Enhancement of antimicrobial peptide activity by metal ions
16 U.S. Pat. No. 6,040,291 Antimicrobial peptide
17 U.S. Pat. No. 6,015,941 Peptide derivatives of tachyplesin having antimicrobial activity
18 U.S. Pat. No. 5,936,063 Antimicrobial peptide isolated from Bufo bufo gargarizans
19 U.S. Pat. No. 5,847,047 Antimicrobial composition of a polymer and a peptide forming amphiphilic helices of the magainin-type
20 U.S. Pat. No. 5,830,993 Synthetic antimicrobial peptide
21 U.S. Pat. No. 5,428,016 Antimicrobial peptide and antimicrobial agent
22 U.S. Pat. No. 5,424,396 Antimicrobial peptide and antimicrobial agent
23 U.S. Pat. No. 5,032,574 Novel antimicrobial peptide
24 U.S. Pat. No. 4,623,733 Antimicrobial peptide

These patents are incorporated herein by reference.

### BRIEF SUMMARY OF THE INVENTION

Embodiments of the present invention are directed to an article comprising a dental implant having a biologically compatible surface. In further embodiments, at least a portion of the surface may further include a therapeutically effective amount of an antimicrobial peptide. The antimicrobial peptide may be immobilized or temporarily attached to the surface of the implant. Preferably, the antimicrobial peptides are alpha helical. More preferably, the antimicrobial peptides are selected from lentivirus lytic peptides (LLPs), lytic base unit peptides (LBUs) and engineered cationic antimicrobial peptides (eCAPs).

In another embodiment, a method for immobilizing antimicrobial peptides on a dental implant may include providing a dental implant, applying a linking layer to at least a portion of a surface of the dental implant, and reacting an antimicrobial peptide to the linking layer.

In another embodiment, an article may include an endodontic instrument. The endodontic instrument may have an elongated shaft and handle portion. The elongated shaft may be covered with at least one layer that includes a filling material and an antimicrobial peptide. The antimicrobial peptide may be immobilized or temporarily attached to the surface of the implant. Preferably, the antimicrobial peptides are alpha helical. More preferably, the antimicrobial peptides are selected from lentivirus lytic peptides (LLPs), lytic base unit peptides (LBUs) and engineered cationic antimicrobial peptides (eCAPs).

In still another embodiment, a method of preventing infection in a subject may include coating at least a portion of a surface of a dental implant with a therapeutically effective amount of an antimicrobial peptide, and implanting the coated dental implant in the subject. The antimicrobial peptide may be immobilized or temporarily attached to the surface of the dental implant.

Another embodiment of the present invention may include a method of preventing and treating infection in a root canal subject that may include coating at least a portion of a surface of an endodontic instrument with a therapeutically effective amount of an antimicrobial peptide, and filling the root canal of a tooth with a filling material, where the filling material may include the antimicrobial peptide and a biocompatible filling material.

### DESCRIPTION OF DRAWINGS

For a fuller understanding of the nature and advantages of the present invention, reference should be had to the following detailed description taken in connection with the accompanying drawings wherein like reference numerals represent like elements throughout, in which:

FIG. 1 depicts an antibacterial peptide covalently bonded to a titanium medical device through an organosilane linking group.

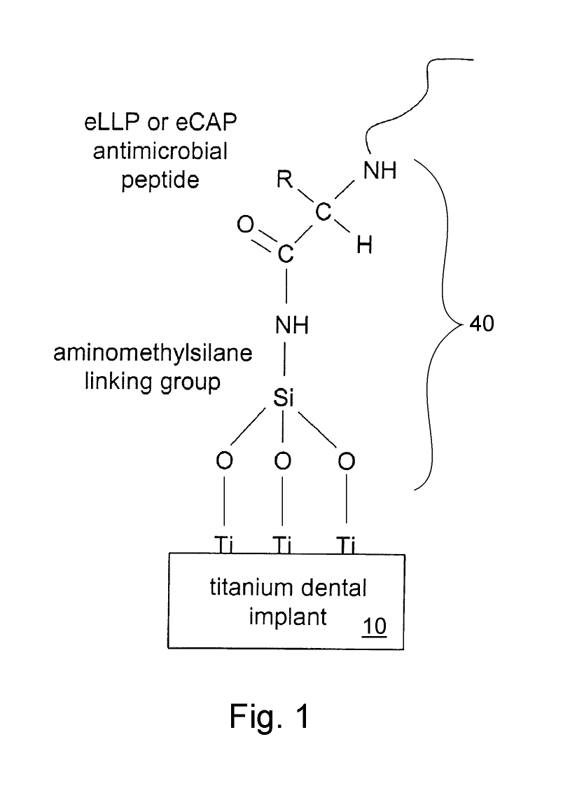

FIG. 2 is a cross sectional elevation view of an embodiment of a one-piece, titanium or titanium alloy or similar biocompatible material dental implant.

FIG. 3 is a cross-sectional elevation of embodiment of FIG. 2 that is covered with a uniform layer of a peptide-based antimicrobial coating that prevents or disrupts bacterial biofilm formation and/or kills the bacteria responsible for infection and re-infection associated with such devices.

FIG. 4 is an elevation view of an embodiment of the elongated shaft and handle portions of the invention.

FIG. 5 is an elevation view of an embodiment of the elongated shaft and handle portions with the elongated shaft's distal portion, i.e., below the stopper, covered with a uniform layer of a filling material, such as gutta-percha or similar thermoplastic polymer material or some composite material, and a uniform layer of a peptide-based antimicrobial.

FIG. 6 is an elevation view of an embodiment of the elongated shaft and handle portions with the elongated shaft's distal portion, i.e., below the stopper, covered with a uniform layer of a filling material, such as gutta-percha or similar thermoplastic polymer material or some composite material, and a uniform layer of a peptide-based antimicrobial covering the uniform layer of filling material and the remainder of the elongated shaft to the proximal end.

FIG. 7 is an elevation view of an embodiment of the elongated shaft and handle portions with a uniform layer of novel compound, comprising gutta-percha or similar thermoplastic polymer material or some composite material, and a peptide-based antimicrobial, covering the distal portion of the elongated shaft below the stopper.

### DETAILED DESCRIPTION OF THE PREFERRED EMBODIMENTS

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “dental implant” is a reference to one or more dental implants and equivalents thereof known to those skilled in the art, and so forth.

As used herein, the term “about” means plus or minus 10% of the numerical value of the number with which it is being used. Therefore, about 50% means in the range of 45%-50%

“Administering” when used in conjunction with a therapeutic means to administer a therapeutic directly into or onto a target tissue or to administer a therapeutic to patient whereby the therapeutic positively impacts the tissue to which it is targeted. Thus, as used herein, the term “administering”, when used in conjunction with antimicrobial peptides, can include, but is not limited to, providing an antimicrobial peptide into or onto the target bacteria from a biomedical device that is coated with an antimicrobial peptide.

As used herein, the term “antimicrobial” refers to the ability of the peptides of the invention to prevent, inhibit or destroy the growth of microbes such as bacteria, fungi, protozoa and viruses.

The terms “dental medical device” as used herein includes an instrument, apparatus, implement, machine, contrivance, implant, or other similar or related article, including a component part, or accessory which is intended for use in the diagnosis of disease or other conditions, or in the cure, mitigation, treatment, or prevention of disease, in man or other animals, or intended to affect the structure or any function of the body of man or other animals. This includes, but is not limited to, for example, dental implants and endodontic instruments, including, but not limited to composites, cores, core pastes, dental fillings for cracks or cavities, crowns, dental bone filling material, resorbable beads and/or sponges for periodontal disease and tooth extraction sites.

The term “improves” is used to convey that the present invention changes either the appearance, form, characteristics and/or the physical attributes of the tissue or bodily fluid to which it is being provided, applied or administered. The change in form may be demonstrated by any of the following alone or in combination: prevention and/or reduction of infections that may accompany root canals, insertion of dental implants and other dental procedures.

The term “inhibiting” includes the administration of a compound of the present invention to prevent the onset of the symptoms, alleviating the symptoms, or eliminating the disease, condition or disorder.

As used herein, the term “peptide” refers to an oligomer of at least two contiguous amino acids, linked together by a peptide bond.

By “pharmaceutically acceptable”, it is meant the carrier, diluents or excipient must be compatible with the other ingredients of the formulation and not deleterious to the recipient thereof.

As used herein, the term “therapeutic” means an agent utilized to treat, combat, ameliorate, prevent or improve an unwanted condition or disease of a patient. In part, embodiments of the present invention are directed to the prevention of infections associated with biomedical devices installed in a subject permanently or for a therapeutic time.

A “therapeutic time” as used herein, is a time required to effectively treat, combat, ameliorate, prevent or improve an unwanted condition or disease of a subject.

A “therapeutically effective amount” or “effective amount” of an antibacterial peptide is a predetermined amount calculated to achieve the desired effect, e.g., to inhibit, block, or reverse the infection of bodily tissue and/or fluids when in the presence of a biomedical device. In embodiments, the effective amount of antibacterial peptide is the amount coated on a biomedical device that prevents the device from being coated with a “biofilm”. A biofilm is an excellent growth medium for bacteria and may ultimately precipitate infection. In other embodiments, the effective amount of an antibacterial peptide is the amount coated on a biomedical device that prevents adhesion of bacteria, subsequent colonization and maturation, and the formation of a biofilm. The specific dose of an antimicrobial peptide in a coating on a biomedical device that is administered according to this invention to obtain therapeutic and/or prophylactic effects will, of course, be determined by the particular circumstances surrounding the case, including, for example, the peptide administered, the nature of the biomedical device, and the contemplated infection to be blocked or treated. A therapeutically effective amount of antimicrobial peptide of this invention is typically an amount such that when it is administered as a component of a biomedical device or a coating on a biomedical device, it is sufficient to achieve an effective inhibition of a biofilm on a biomedical device.

The terms “treat,” “treated,” or “treating” as used herein refers to both therapeutic, treatment and prophylactic or preventative measures, wherein the object is to prevent or slow down (lessen) a bacterial infection, or to obtain beneficial or desired clinical results, such as, but not limited to prevention of infection and re-infection associated with an endodontically prepared and treated root canal. For the purposes of this invention, beneficial or desired clinical results include, but are not limited to, alleviation of symptoms; diminishment of the extent of the condition, disorder or disease; stabilization (e.g., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

Generally speaking, the term “tissue” refers to any aggregation of similarly specialized cells which are united in the performance of a particular function.

Embodiments of the present invention are directed to medical devices coated with antimicrobial peptides. In preferred embodiments, the medical devices are permanently or semi-permanently introduced into the body of a subject, preferably a human. In preferred embodiments, the medical devices are selected from dental implants, dental fillings, and endodontic instruments. Preferably, the antimicrobial peptides are alpha helical peptides. More preferably, the antimicrobial peptides comprise peptides selected from linear lytic peptides (LLPs), lytic base unit peptides (LBUs), and engineered cationic peptides (eCAPs). The antimicrobial peptide may be immobilized or temporarily attached to the surface of the dental implants, dental fillings, and endodontic instruments, including, but not limited to composites, cores, core pastes, posts, dental fillings for cracks or cavities, crowns, dental bone filling material, resorbable beads and/or sponges for periodontal disease and tooth extractions.

Further embodiments of the present invention are directed to methods of preventing or treating an infection comprising implanting an implantable dental medical device coated with an antimicrobial peptide into a subject.

Further embodiments of the present invention are directed to coating antimicrobial peptides on medical devices, preferably dental implants, dental fillings and endodontic instruments, including, but not limited to composites, cores, core pastes, dental fillings for cracks or cavities, crowns, dental bone filling material, resorbable beads and/or sponges for periodontal disease and tooth extractions.

Preferably, the antimicrobial peptides are alpha helical peptides. More preferably, the antimicrobial peptides comprise peptides selected from linear lytic peptides (LLPs), lytic base unit peptides (LBUs), and engineered cationic peptides (eCAPs). The antimicrobial peptide may be immobilized or temporarily attached to the surface of the dental implants, dental fillings, and endodontic instruments.

Antimicrobial peptides that are useful in embodiments of the present invention include those derived from selected amino acid sequences in viral transmembrane proteins. In particular, the peptides may be derived from lentiviruses, primarily human immunodeficiency virus (HIV), simian immunodeficiency virus (Sly), and equine infectious anemia virus (EIAV). These are lytic peptides derived from lentiviruses and are designated by the term “LLPs” as disclosed in U.S. Pat. No. 5,714,577, which is incorporated herein in its entirety. In contrast to other antimicrobial peptides, which are specifically encoded by their own genes, LLPs are unique in that they are derived from naturally occurring sequences that are part of a larger folded protein.

In another embodiment of the invention, the antimicrobial peptides useful in embodiments of the present invention are structural and functional analogs of the naturally occurring parent peptides which exhibit selective toxicity for microorganisms.

As used herein, the term “analog” refers to a peptide which contains substitutions, rearrangements, deletions, additions and/or chemical modifications in the amino acid sequence of parent peptide, and retains the structural and functional properties of the parent peptide.

In another embodiment of the invention, the antimicrobial peptides useful in' embodiments of the present invention are structural and functional homologs of the naturally occurring parent peptides which exhibit selective toxicity for microorganisms.

As used herein, the term “homolog” refers to a peptide, the sequence of which is at least 80% homologous to the amino acid sequence of a parent peptide, and retains the structural and functional properties of the parent peptide.

The amino acid sequences of the antimicrobial peptides useful in embodiments of the invention correspond to or are analogous to or homologous to peptides LLPI and LLP2, which, in turn, correspond to residues 828-855 and 768-788 of the HIV-1 TM protein (gp41) (strain HXB2R), respectively; peptides SLP-1, SLP-2A and SLP-2B (SLP2 region), which, in turn, correspond to residues 852-879, 771-795 and 790-817 of the SIV TM protein (MM239 strain of SIV), respectively; and peptide ELP, which corresponds to residues 808-836 of EIAV (Wyoming strain). The striking feature of these sequences is their lack of sequence homology to known cytolytic peptides (e.g. magainins); however, each is rich in positively charged residues and is predicted to form an amphipathic helix. This structure imparts to each of the peptides a unique but potent antimicrobial activity.

The antimicrobial peptides useful in embodiments of the invention are unique in their functional properties. In general, cytolytic peptides can be classified into two major functional types. Antibacterial peptides (magainins and cecropins, for example) specifically kill bacteria. Hemolytic peptides, on the other hand, generally both kill bacteria and lyse red blood cells; melittin from bee venom is an example of such a peptide. The antimicrobial peptides useful in the present invention are moderately hemolytic; they do lyse red blood cells, but only at high concentrations. The unique structure of the antimicrobial peptides of the invention imparts high potency while maintaining selectivity. The structural properties defining the antimicrobial peptides useful in embodiments of the present invention include, inter alia, a significant number of positively charged amino acid residues and the ability to form three-dimensional amphipathic helical structures. Functional properties include, inter alia, a selective antimicrobial cytolytic activity, but minimal cytolytic activity toward mammalian cells.

The structural formulae of the exemplary antimicrobial peptides useful in embodiments of the present invention corresponding to regions of TM proteins derived from HIV-1, SIV, and EIAV are listed in TABLE 1.

TABLE 1
Parent Antimicrobial Peptides

| Peptide | | |
|---|---|---|
| Name | Amino Acid Sequence | Sequence Source |
| LLP1 | RVIEVVQGACRAIRHIPRR | HIVHXB2R 828-855 |
| (SEQ ID NO. 1) | IRQGLERIL | |
| LLP2 | YHRLRDLLLIVTRIVELLG | HIVHXB2R 768-788 |
| (SEQ ID NO. 2) | RR | |
| SLP1 | DLWETLRRGGRWILAIPRR | SIVMM239 852-879 |
| (SEQ ID NO. 3) | IRQGLELTL | |
| SLP2A | FLIRQLIRLLTWLFSNCRT | SIVMM239 771-795 |
| (SEQ ID NO. 4) | LLSRVY | |
| SLP2B | LLSRVYQILQPILQRLSAT | SIVMM239 790-817 |
| (SEQ ID NO. 5) | LQRIREVLR | |
| ELP | RIAGYGLRGLAVIIRICIR | EIAV 808-836 |
| (SEQ ID NO. 6) | GLNLIFEIIR | |

wherein: A = Ala = Alanine R = Arg = Arginine N =
Asn = Asparagine D = Asp = Aspartic acid B = Asx =
Asparagine or aspartic acid C = Cys = Cysteine Q =
Gln = Glutamine E = Glu = Glutalmic acid Z = Glx =
Glutamine or glutamic acid G = Gly = Glycine H =
His = Histidine I = Ile = Isoleucine L = Leu =
Leucine K = Lys = Lysine = Met = Methionine F =
Phe = Phenylalanine P = Pro = Porline S = Ser =
Serine T = Thr = Threonine W = Trp = Tryptophan Y =
Tyr = Tyrosine V = Val = Valine
¹ Sequences of the family of LLPs derived from HIV and SIV envelope proteins are consistent with the numbering in Myers. The sequence of ELP, the peptide derived from the ENV protein of EIAV, is from the Wyoming strain (Rushlow et al., 1986).

A peptide analog or homolog useful in embodiments of the present invention may be identified by the following criteria: (1) the parent peptide of the analog is an antimicrobial peptide having a sequence which corresponds to a vital TM protein, particularly a lentivirus TM protein; (2) the amino acid sequence of the peptide is capable of forming an amphipathic helix and contains a number of positively charged residues; (3) the peptide is selectively antimicrobial in its biological function and has minimal cytolytic activity toward mammalian cells.

In the design of the peptide analogs of the antimicrobial peptides useful in embodiments of the present invention, the allowed amino acid interchanges which are contemplated include, inter alia, the substitution of an individual residue in the peptide with a residue that falls within the same chemical subset, e.g., a hydrophobic amino acid replaced by the same or a positively charged residue with the same. This degree of substitution allows for the construction of peptide analogs from the parent structure which retain the structural and functional properties of the parent peptide, without undue experimentation.

Analogs may also contain non-conservative amino acid interchanges provided that structural and functional properties are retained or enhanced. A singular characteristic of the antimicrobial peptides useful in embodiments of the present invention is the presence of a significant number of positively charged residues, especially arginine. Analogs and homologs include those that encompass substitutions which retain the overall charge characteristics of the peptides. Preferably, the peptides useful in embodiment of the present invention have a net charge of at least +3 at neutral pH. Net charge is calculated by adding the sum of the charge value of positively charged amino acids (arginine, lysine, histidine) (+1) and the charge value of the negatively charged amino acids (aspartic acid, glutamic acid) (−1). Thus, the positively charged arginines in the peptides may be substituted by histidine or lysine so as to retain positively charged residues. Analogs may be designed which increase the number of positively charged amino acids so long as the antimicrobial activity of the peptide is not diminished, for example, the number of arginine residues may be increased. An analog which increased the number of positive charges in an LLPI analog peptide has been shown to be more toxic to bacteria than the parent LLP1.

Additional analogs of the peptides useful in embodiments of the present invention can have an altered number of hydrophobic amino acids based on the parent peptide, producing peptides having altered specificity. For example, an increase in hydrophilic residues appears to reduce antimicrobial effectiveness. However, such changes appear to increase antimicrobial specificity by reducing undesired hemolytic activity. Therefore, based on the teachings and guidance herein, one skilled in the art can design analogs useful in embodiments of the invention which have a desired potency and selectivity.

In the design of peptide homologs of the antimicrobial peptides useful in embodiments of the invention, the amino acid changes which are contemplated include, inter alia, the replacement of amino acid residues in a parent peptide such that the homologous peptide retains the structural and functional properties of the parent peptide.

A primary common and recognizable feature of the antimicrobial peptides of the present inventions is their secondary structure, or more specifically, their potential to form amphipathic structures, which may be in the form of an alpha-helix or a beta conformation. An alpha-helix motif, for example, comprises residues arranged such that 3.5 amino acid residues complete 1 turn of the helix. An estimate of amphipathicity may therefore be made by examination of the amino acid sequence; for example, peptides comprising amino acid residues arranged in a hydrophobic-hydrophobic-hydrophilic-hydrophilic repeating motif are highly likely to form an alpha-helix. Amino acid residues arranged to alternate in a hydrophobic-hydrophilic-hydrophobic-hydrophilic repeating motif are likely to form a beta conformation. Such “ideal” motifs are found in the antimicrobial peptides of the invention and as such may be used by those skilled in the art as a foundation for engineering additional amphipathic peptide analogs of the invention without great difficulty based on the teachings herein. The antimicrobial peptides useful in embodiments of the invention may further contain proline or glycine, amino acid residues which can be tolerated within a general amphipathic structure and may indicate demarcations between different amphipathic regions. These residues may impart a structure which enhances the activity and selectivity of a peptide because of a bend or kink between helices. For example, a solidly helical structure may be less selective (e.g. LLP2, SLP2A). Homologs may also be engineered, using these structural considerations that are at least 80% homologous to the amino acid sequence of a parent peptide, and retain the structural and essential antimicrobial functional properties of the parent peptide.

Analogs and homologs in which the amphipathicity of a peptide is increased by additions, deletions and/or substitutions of amino acids in a parent peptide are within the scope of the invention.

Analogs and/or homologs of the antimicrobial peptides useful in embodiments of the present invention preferably contain at least one cysteine which, by virtue of its capacity to form a disulfide bond, can confer high potency and a very high degree of bactericidal activity to a peptide containing such a residue. A peptide preferably contains a single cysteine residue to ensure that any disulfide bond formed by the cysteine would be intermolecular and result in a disulfide-inked dimeric peptide (e.g. bis-LLP1). The residue to be replaced by cysteine is preferably neither very hydrophobic nor basic and lies on the interface of the hydrophilic and hydrophobic faces of the amphipathic structure when modeled as such. Computer modeling programs such as “Helical wheel” may be used to design such peptides.

The antimicrobial peptides useful in embodiments of the present invention generally comprise a positively charged C-terminus. However, those peptides having this characteristic generally have some hemolytic activity, and analogs which optimize antimicrobial selectivity (i.e., decrease hemolytic activity) may be those which replace the positively charged C-terminus with negatively charged or hydrophobic residues. Since reduction of the basic character of the C-terminus may provide antimicrobial selectivity, analogs are provided in which the amino acids of the C-terminus region may be reversed in situ, or, alternatively, the N-terminus and C-terminus regions may be interchanged. The peptide is then comprised of a positively charged N-terminus and a hydrophobic C-terminus.

Analogs and homologs which are chimeras of particular antimicrobial peptides and/or other cytolytic peptides are useful in embodiments of the present invention, provided that the structural and functional properties described herein are retained.

In another embodiment of the invention, the use of D-amino acids in place of L-amino acids in the peptides may provide increased metabolic stability, since peptides containing D-amino acids are resistant to mammalian proteases, which generally cleave peptides composed of L-amino acids.

Embodiments of the present invention may also include the use of peptide analogs and homologs which are truncated, i.e., shorter than the parent amino acid sequence or to truncated parent peptide fragments. A minimal length required to effectuate ion-channel formation in membranes is believed to be a peptide of 8-12 amino acid residues in length. It has been suggested that the antimicrobial peptides may dimerize so as to comprise the approximately 20 amino acid length believed to be required to transverse a membrane. As discussed above, the inclusion of a cysteine residue in an antimicrobial peptide is of importance in facilitating the formation of intramolecular or intermolecular disulfide bonds which can stabilize a dimeric peptide. A 21-amino acid segment of LLP1 was capable of pore formation in planar lipid bilayers in vitro, although it was not tested for antimicrobial activity. The design of analogs of minimal length can optimize potency of the peptides in terms of effectiveness per mass.

The following analogs and homologs, derived from the parent peptides shown in TABLE 1, are also exemplary of the antimicrobial peptides useful in embodiments of the present invention, and have the following primary structural formulae:

LLPI ANALOGS:

SEQ ID NO. 7, and SEQ ID NO. 10 through SEQ ID NO. 72;

SLP-1 ANALOGS:

SEQ ID NO. 73 through SEQ ID NO. 107.

LLP2 ANALOGS:

SEQ ID NO. 108 through SEQ ID NO. 145.

SLP2B ANALOGS

SEQ ID NO. 146 through SEQ ID NO. 151.

SLP2 REGION ANALOGS

SEQ ID NO. 152 through SEQ ID NO. 154.

ELP ANALOGS

SEQ ID NO. 155 through SEQ ID NO. 159.

Preferably, the antimicrobial peptides of the invention have the following structural formula: SEQ ID NO. 4; SEQ ID NO. 5; SEQ ID NO. 7, SEQ ID NO. 8 and SEQ ID NO: 9.

Analogs and homologs of other naturally occurring lytic peptides derived from other lentivirus proteins are also within the scope of the invention. Peptides may be derived from proteins of any lentivirus or any DNA or RNA virus including, but not limited to, HIV-1, HIV-2, SlY, EIAV, feline immunodeficiency virus (FIV), bovine immunodeficiency virus (BIV), visna virus and all clades, subclasses and isolates thereof.

In another embodiment of the invention, antimicrobial peptides which are derived from, and are analogs of, the LLPI peptide parent sequence corresponding to amino acids 828856 of the HIV-1 viral isolate HXB2R Env and include SA-5 (SEQ ID NO. 1), LSA-5 (SEQ ID NO: 2) and WLSA-5 (SEQ ID NO. 3) (see Table 1 below) are useful to coat medical devices. The antimicrobial activity of other LLPI peptide analogues has been previously described (see Tencza et al., 1999, Journal of Antimicrobial Chemotherapy 44:33-41, U.S. Pat. No. 5,714,577 of Montelaro et al. and U.S. Pat. No. 5,945,507 of Montelaro et al.). Antimicrobial peptides useful in this invention include those disclosed in U.S. Pat. No. 6,887,847, which is incorporated herein in its entirety. In embodiments of the invention, the antimicrobial peptides are LLPI analogs having modifications based on the following principles: (i) optimizing amphipathicity, (ii) substituting arginine (Arg) on the charged face and/or valine (Val) or tryptophan (Trp) on the hydrophobic face with another amino acid, and (iii) increasing peptide length (referred to collectively herein as Lytic Base Unit peptides (LBU peptides), e.g. LBU-2, (SEQ ID NO. 174); LBU-3, (SEQ ID NO. 175); LBU-3.5, (SEQ ID NO. 176); LBU-4, (SEQ ID NO. 177); WLBU1, (SEQ ID NO. 178), WLBU-2, (SEQ ID NO. 179), WLBU-3, (SEQ ID NO. 180); and WLBU-4, (SEQ ID NO. 181); see Table 2). The LBU peptides deviate greatly from the parent LLPI, for example, LBU-2 and LBU-3 deviate from the parent LLPI sequence by greater than 90%.

TABLE 2

| | | |
|---|---|---|
| SA-5 | RVIRV VQRAC RAIRH IVRRI | (SEQ ID NO. 170) |
| | RQGLR RIL | |
| LSA-5 | RVIRV VQRAC RAIRH IVRRI | (SEQ ID NO. 171) |
| | RQGLR RILRV V | |
| WLSA-5 | RWIRV VQRWC RAIRH IWRRI | (SEQ ID NO. 172) |
| | RQGLR RWLRV V | |
| LBU-1 | RVVRV VRRVV RR | (SEQ ID NO. 173) |
| LBU-2 | RRVVR RVRRV VRRVV RVVRR | (SEQ ID NO. 174) |
| | VVRR | |
| LBU-3 | VRRVV RRVVR VVRRV VRRVR | (SEQ ID NO. 175) |
| | RVVRR VVRVV RRVVRR | |
| LBU-3.5 | RRVVRRVRRVVRRVVRVVRRVVRRVR | (SEQ ID NO. 176) |
| | RVVR RVVRV VRRVV RR | |
| LBU-4 | RVVRV VRRVV-RRVRR VVRRV | (SEQ ID NO. 177) |
| | VRVVR RVVRR VRRVV RRVVR | |
| | VVRRV VRR | |
| WLBU-1 | RVVRV VRRWV RR | (SEQ ID NO. 178) |
| WLBU-2 | RRWVR RVRRV WRRVV RVVRR | (SEQ ID NO. 179) |
| | WVVRR | |
| WLBU-3 | VRRVW RRVVR VVRRW VRRVR | (SEQ ID NO. 180) |
| | RVWRR VVRVV RRWVR R | |
| WLBU-4 | RVVRV VRRWV RRVRR VWRRV | (SEQ ID NO. 181) |
| | VRVVR RWVRR VRRVW RRVVR | |
| | VVRRW RVV | |

The LLPI analogue peptides and the LBU peptides (collectively referred to herein as “engineered LLPs” (eLLPs)) useful in embodiments of the present invention have a broader spectrum of activity (i.e., the ability to kill highly resistant bacteria) and increased potency (i.e., lowering the molar concentration required to kill bacteria) when compared with previously described LLPI analogs. The eLLPs of the present invention are highly inhibitory to microorganisms under physiologic salt concentrations, function in the presence of synovial fluid, and demonstrate only minimal toxicity in animal models. As a result, the eLLPs may be defined as selective antimicrobial agents. In addition, the peptides useful in embodiments of the present invention function by disrupting bacterial membranes and are active when bound to a solid phase. The ability of these peptides to maintain activity when bound to a solid phase is a significant advantage over conventional antibiotics.

The antimicrobial peptides useful in embodiments of the present invention, collectively referred to herein as “eLLPs”, exhibit antimicrobial activity against diverse microorganisms, and are analogs of the LLPI peptide corresponding to amino acids 828-856 of the HIV-1 viral isolate HXB2R Env TM. The eLLPs comprise Arg-rich sequences, which, when modeled for secondary structure, display high amphipathicity and hydrophobic moment. The eLLPs are highly inhibitory to microorganisms, but significantly less toxic to mammalian cells. As a result, these peptides can be characterized as selective antimicrobial agents. In addition, the eLLPs of the present invention include LLPI peptide analogs comprising modifications based on the following principles: (i) optimizing amphipathicity, (ii) substituting Arg on the charged face and/or Val or Trp on the hydrophobic face, and (iii) increasing peptide length, collectively referred to herein as LBU peptides.

Accordingly, one embodiment of the present invention provides a dental medical device comprising a dental medical device coated with an antimicrobial peptide. In a preferred embodiment, the antimicrobial peptide is selected from LLPs, LBUs and eCAPs. Preferably, the antimicrobial peptide is selected from SEQ ID NO. 1 through SEQ ID NO. 185. The antimicrobial peptide may be immobilized or temporarily attached to the surface of the dental medical device.

In a preferred embodiment, the antimicrobial peptides are present on the dental medical device in a therapeutically effective amount. The dental medical device may be any device that is implanted into or onto the body, preferably, for example, a dental implant, dental filling material, and endodontic instruments, including, but not limited to composites, cores, core pastes, dental fillings for cracks or cavities, crowns, dental bone filling material, resorbable beads and/or sponges for periodontal disease and tooth extraction sites.

In certain embodiments the antimicrobial peptide is coated on the dental medical device such that the antimicrobial peptides substantially cover the entire surface of the device. In certain embodiments, the antimicrobial peptide is coated on the dental medical device such that the antimicrobial peptide covers a partial surface or portion of the surface of the device, such as, for example, the portion of the device that it inserted into or placed on the body of the subject. In further embodiments, the antimicrobial peptide is coated on the dental medical device and further includes a second therapeutic agent.

In one preferred embodiment, the antimicrobial peptides useful in the present invention have the following structural formula: SEQ ID NO. 4, SEQ ID NO. 5, SEQ ID NO. 8, SEQ ID NO. 9, SEQ ID NO. 173, SEQ ID NO. 174, SEQ ID NO. 175, SEQ ID NO. 176, SEQ ID NO. 177, SEQ ID NO. 178, SEQ ID NO. 179, SEQ ID NO. 180, SEQ ID NO. 181.

An embodiment of a medical device of the instant invention includes dental implants. They function as an artificial root or anchor for an artificial tooth. Dental implants obtain their stability by being embedded in the patient's jawbone, providing the artificial tooth with an anchor. The neck of a dental implant protrudes above the patient's periodontal tissue line.

Infection is a leading cause of implant failure. A dental implant infection is serious and appropriate antibiotic treatment should begin immediately. The mouth contains harmful bacteria that surround periodontal tissues and these bacteria can cause an infection around the implant. Infection can occur around newly or previously placed implant and can cause loss of supporting bone or damage to the protective periodontal tissues. Repair of an infected implant is painful, time consuming, and often unsuccessful. The use of antibiotics and debridement of the periodontal tissue area is part of the treatment protocol. Despite this treatment protocol, many dental implants are lost due to infection.

Embodiments herein are directed towards an improved one-piece, titanium, titanium alloy or similar biocompatible material, single or multiple, tooth dental implant having a peptide-based antimicrobial coating that prevents or disrupts bacterial biofilm formation on the surface of the implant and/or kills the bacteria responsible for infection and re-infection associated with such devices.

Preferred antimicrobial based peptides for coating on dental implants include, but are not limited to, LLPs, LBUs, and eCAPs, as presented above, including preferably, SEQ ID NO: 4, SEQ ID NO. 5, SEQ ID NO. 8, SEQ ID NO. 9, SEQ ID NO. 173, SEQ ID NO. 174, SEQ ID NO. 175, SEQ ID NO. 176, SEQ ID NO. 177, SEQ ID NO. 178, SEQ ID NO. 179, SEQ ID NO. 180, SEQ ID NO. 181.

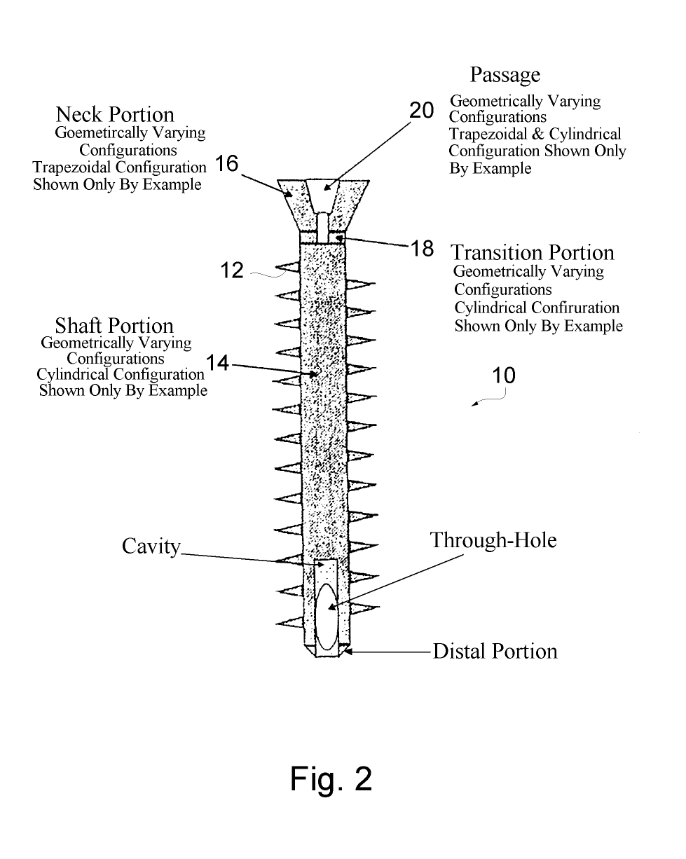

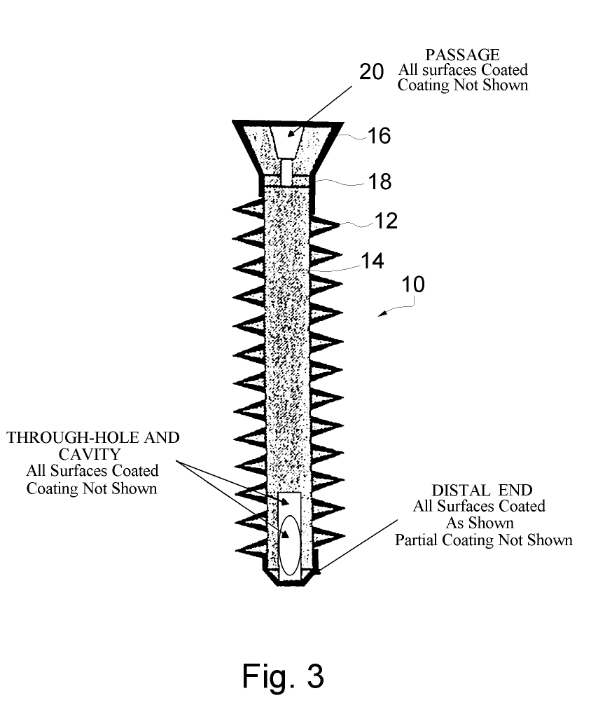

A preferred embodiment of the dental implant invention is that depicted in FIG. 2. In a useful embodiment of the present invention, a uniform layer of peptide-based antimicrobial uniformly covers over all surfaces, as shown in FIG. 3, which prevents or disrupts bacterial biofilm formation and/or kills the bacteria responsible for infection and reinfection associated with dental implants.

In other useful embodiments of the present invention, a uniform layer of peptide based antimicrobial may be applied to the self-tapping threads of a shaft portion of a dental implant. This portion consists of varying degrees of cylindrical or tapering shapes and degrees of roughness and may be grit-blasted with bio-compatible material such as titanium plasma spray (TSP) or hydroxylapetite (HA) to promote osteointegration between the bone and implant.

In another embodiment, a uniform layer of peptide-based antimicrobial applied to the geometrically varying neck portion abutting the self-tapping threaded shaft section at the proximal end that will minimize or eliminate adherence of bacteria and dental plaque.

In another embodiment, a uniform layer of peptide-based antimicrobial may be applied to the transition portion between the neck and threaded shaft portions.

Yet in another embodiment of the present invention, a uniform layer of peptide based antimicrobial may be applied to the geometrically varying threaded or unthreaded passage within the neck and transition portions. In embodiments of the present invention, these may be coated with a uniform layer of peptide-based antimicrobial and may be configured geometrically to accommodate connection with an abutment or human prosthetic tooth.

Other embodiments of the invention disclosed and claimed include an endodontic instrument coated with a peptide-based antimicrobial for use in root canal therapy. In embodiments, preferred antimicrobial based peptides for coating on endodontic instruments include, but are not limited to, SEQ ID NO. 4, SEQ ID NO. 5, SEQ ID NO. 8, SEQ ID NO. 9, SEQ ID NO. 173, SEQ ID NO. 174, SEQ ID NO. 175, SEQ ID NO. 176, SEQ ID NO. 177, SEQ ID NO. 178, SEQ ID NO. 179, SEQ ID NO. 180, SEQ ID NO. 181.

Embodiments herein include, but are not limited to an antimicrobial peptide coating, as disclosed hereinabove, on an endodontic instrument. In these embodiments, the antimicrobial coating substantially covers the entire surface of the endodontic instrument. Still other embodiments include a partial antimicrobial coating for an endodontic instrument. In these embodiments, the coating may only cover a portion of the surface of the endodontic instrument. The portion of the device that is coated may include only a portion that is inserted into a patient's mouth. Still further embodiments of the present invention include an endodontic instrument that is coated with a therapeutically effective amount of antimicrobial peptide. The antimicrobial peptide may be immobilized or temporarily attached to the surface of the endodontic instrument. Another embodiment of the present invention includes methods to coat an endodontic instrument with antimicrobial peptides. Preferred methods for coating an endodontic instrument with antimicrobial peptides are the same as those for coating a medical device and are disclosed hereinabove.

In another embodiment, the invention may include an endodontic instrument coated with a peptide-based antimicrobial for use in root canal therapy that includes: a) an elongated shaft, having a proximal and distal end with increasingly tapering blade and a sliding stopper; b) a conventional handle mounted to the proximal end for manipulation by a dentist; and a c) coating consisting of: i) a uniform layer of a filling material, such as gutta-percha or similar thermoplastic polymer material or some composite or resin material, covered by a uniform layer of a peptide-based antimicrobial; or ii) a uniform layer of novel compound, comprising a filling material, such as gutta-percha or similar thermoplastic polymer material or some composite material, and a peptide-based antimicrobial.

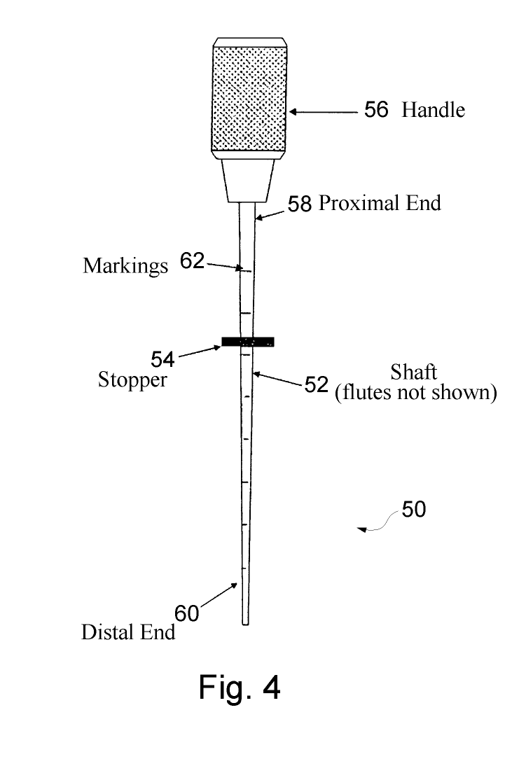

In an embodiment, the elongated shaft is of circular cross-section and has a sliding stopper between its proximal and distal ends (FIG. 4). It can be comprised of plastic, nickel-titanium alloy, or stainless steel. The shaft is tapered with varying diameters and calibrated with conventional depth markings. The shaft can be grooved with helical, triangular, or quadrangular flutes along its entire working length. The shaft's distal end is located near the apical region of the root and is the end of the instrument's working length. An embodiment of an elongated shaft is shown in FIG. 4.

In an embodiment, the elongated shaft may have a conventional handle affixed to the proximal end for a dentist to engage the shaft between the thumb and forefinger for manipulation of the shaft, i.e., pushing, rotating, etc. The handle is of a diameter larger than that of the elongated shaft and is integral to the shaft at the proximal end. The handle can be of plastic or metal material. An embodiment of the handle is shown in FIG. 4.

In another embodiment, the elongated shaft has a washer-like stopper which slidably receives shaft along the working length of the shaft and can be manually operated to retain coating materials when the shaft is withdrawn. An embodiment of the stopper is shown in FIG. 4.

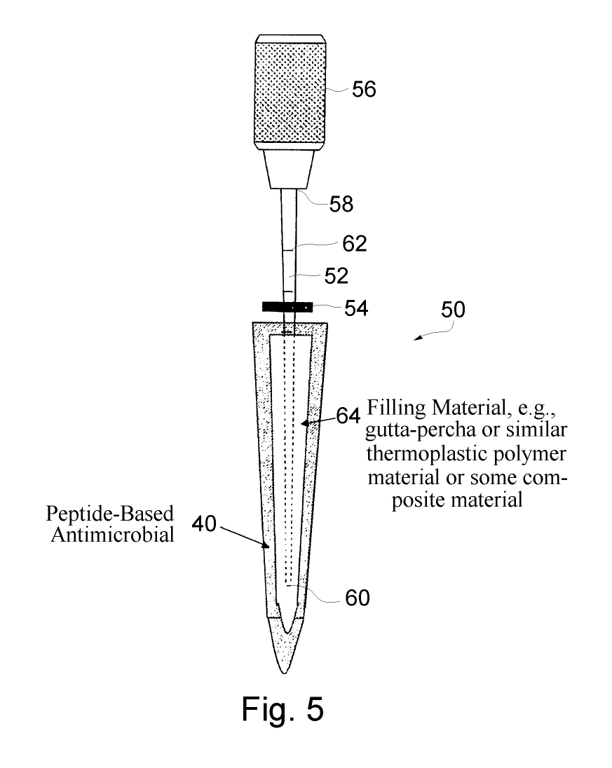

In an embodiment, the elongated shaft has a uniform layer of a filling material, such as gutta-percha or similar thermoplastic polymer material or some composite material, applied around the circumference of the elongated shaft for a working length from the distal end to the stopper. An exemplary embodiment of the filling material layer is shown in FIG. 5. The working length of elongated shaft and layer of a filling material, such as gutta-percha or similar thermoplastic polymer material or some composite material, below the stopper has a uniform layer of peptide-based, antimicrobial applied around the circumference of that working length. A preferred embodiment of this endodontic instrument coated with a peptide-based antimicrobial invention is shown in FIG. 5.

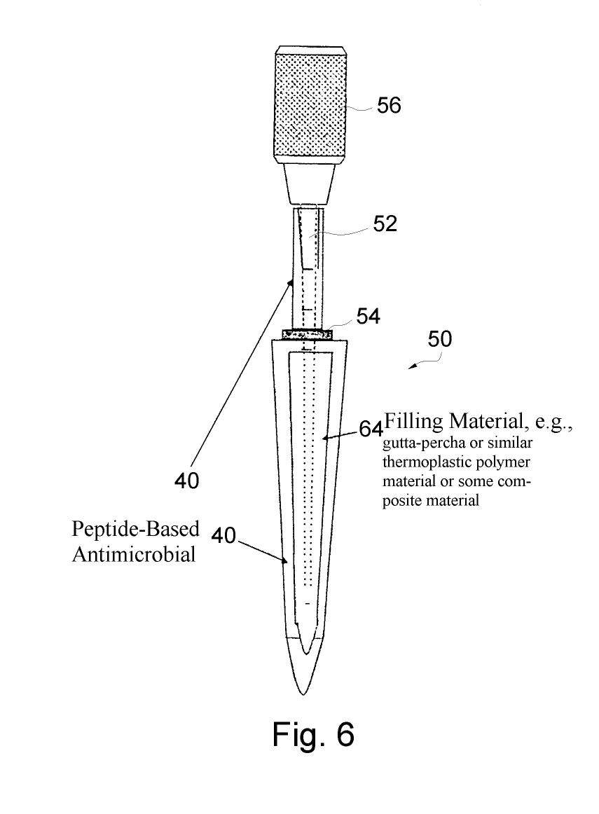

In an embodiment, an elongated shaft can also have a uniform layer of a filling material, such as gutta-percha or similar thermoplastic polymer material or some composite material, applied around the circumference of the elongated shaft for a working length from the distal end to the stopper, and a uniform layer of a peptide-based antimicrobial applied around the circumference of that working length and the remainder of the elongated shaft to the proximal end. An embodiment of this invention is shown in FIG. 6.

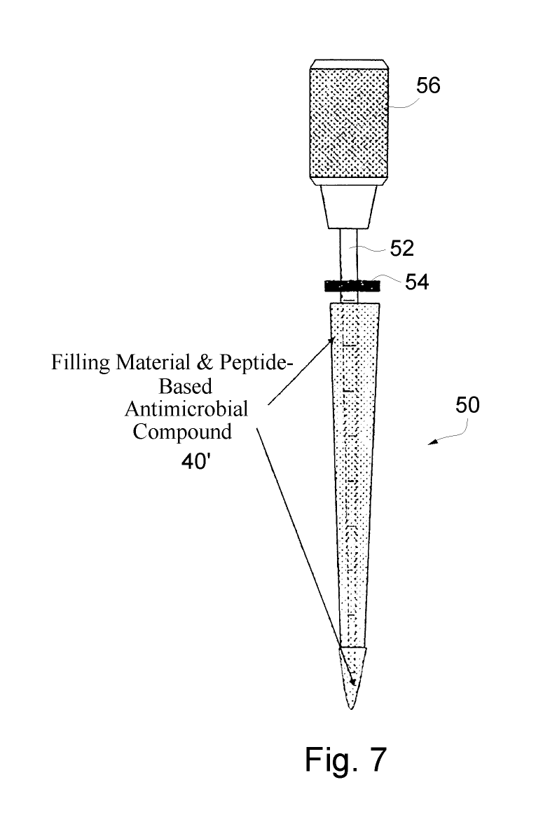

An elongated shaft can also have a uniform layer of a compound comprised of gutta-percha or similar thermoplastic polymer material or some composite material and a peptide-based antimicrobial mixture, applied around the circumference of the elongated shaft for a working length from the distal end to the stopper. An embodiment of this invention is shown in FIG. 7.

Embodiments of the present invention are directed to bonding and immobilizing antibacterial peptides to biocompatible material surfaces of permanent or semi-permanent implantable dental medical devices for mammals. Permanent or semi-permanent implantable dental medical devices may include but are not limited to for example dental implant and dental filling material. In other embodiments, the antibacterial peptides may be bonded and immobilized on surfaces of biomedical devices that may invade the normal body barriers for only a therapeutic time. Other such devices include but are not limited to endodontic instruments, for example.

The antimicrobial peptides of the coating of the present invention can also be administered in combination with other active ingredients, such as, for example, bone growth promoters, adjuvants, protease inhibitors, or other compatible drugs or compounds where such combination is seen to be desirable or advantageous in achieving the desired effects of the methods described herein. These additional active ingredients or agents may be combined with the antimicrobial peptide or may be a second layer coating the device.

In embodiments, the antimicrobial peptides on biomedical device surfaces may prevent the formation of a biofilm on the dental medical device surface. A biofilm is an excellent growth medium for bacteria and precipitates infection. By prevention of bacterial colonization of the dental medical device surface, any bacteria remaining in the subjects system are accessible for clearance by the patient's immune system. The antimicrobial peptide may be immobilized or temporarily attached to the surface of the device.

A biocompatible material (or biomaterial) is a synthetic or natural material used to replace part of a living system or to function in intimate contact with living tissue. Biocompatible materials are intended to interface with biological systems to evaluate, treat, augment or replace any tissue, organ or function of the body. Biocompatible materials may include for example synthetic and natural polymers, such as but not limited to acrylic, polyethylene terephthalate (PET), polymethyl methacrylate (PMMA), polypropylene resins cellulose, gutta-percha, and collagen. Metals, such as precious metals used in dental fillings, and metals with high strength, low modulus and body fluid resistance, such as, but not limited to titanium and titanium alloys are considered biocompatible materials. For biomedical devices that require flexibility, metals such as nitinol, other alloys of nickel titanium, stainless steel, and cobalt chromium are exemplary biomaterials encompassed in embodiments herein. Any biomaterials known now or hereafter to one of ordinary skill in the art are encompassed in embodiments herein.

The antimicrobial peptides may be bonded directly to at least a portion of a biocompatible surface of a dental medical device, or alternatively may be bonded to linking groups that are bonded to at least a portion of the biocompatible surface. For immobilization of the antimicrobial peptide on the dental medical device surface, covalent bond formation between the surface and the peptide; or between the surface, the linking group, and the peptide is preferred. Other types of bonding, including, but not limited to acid-base interactions, ionic bonding, hydrogen bonding, dispersion forces, and Van Der Waals interactions are included in the scope of embodiments herein.

The term “immobilized” as used herein refers to antibacterial peptides that remain bonded to the surface of the device, and maintain efficacy for at least a therapeutic time period. The peptide remains bonded and active as a bactericide at least until the chance for bacterial infection is not elevated as compared to the condition of the patient's normal physiology. For embodiments that include permanent or semi-permanent implantable biomedical devices, the peptide may remain bonded to the surface of the device after the threat of infection has passed in order to promote the adhesion and maturation of cells on the implanted device.

In embodiments where the antibacterial peptide is bonded directly to the surface of the dental medical device the biomedical device may be cleaned, passivated, or activated to increase the polarity of the surface, or to provide functional groups on the surface, to promote chemical reaction of the peptide and the surface. Cleaning, passivating, or activating may include chemical processes known now or hereafter to one of ordinary skill in the art, such as, but not limited to, repetitive acid or alkaline washing followed by rinsing with distilled or deionized water. Cleaning, passivating, or activating a surface may also include physical processes such as for example corona discharge. Any cleaning and/or activating process known now or hereafter to one of ordinary skill in the art is within the scope of embodiments herein.

In embodiments, a linking group, also known as a linker, a coupling agent, a primer, and a tie layer, is used to bond an antimicrobial peptide to a dental device surface and is a form of chemical surface functionalization. One end of the linking group readily forms bonds that are stable under physiological conditions with the device surface. The linking group may then be capable of forming hydrolytically and physiologically stable bonds with a terminal end of an antimicrobial peptide. In other embodiments, after the surface is functionalized with a linking group, the linking group may be further treated to create reactive sites on the linking group. Such treatments may include corona discharge, plasma processes, flame treatments, or other processes known now or hereafter to one of ordinary skill in the art to create polar and/or reactive groups, such as for example, hydroxyl carboxyl and carbonyl, on a non-polar linking group. In this fashion, the terminal ends of the antimicrobial peptide can chemically bond with the linking group.

In other methods a material such as a block copolymer may be used to functionalize a surface of a dental medical device with antimicrobial proteins. In theses instances, a hydrophobic block of a copolymer may interact with a hydrophobic device surface. A hydrophilic block of the copolymer may extend away from the surface, and can have functional groups that can react with the surface.

In other embodiments, a parylene coating may be initially applied to the dental medical device surface. Parylene is the tradename for a variety of polyxylylene polymers marketed by Para Tech Coating, Inc., Aliso Viejo, Calif. Parylene C is a polymer manufactured from di-p-xylene, a dimer of p-xylene. Di-p-xylene, more properly known as [2.2] paracyclophane, is made from p-xylene in several steps involving bromination, amination and elimination. Heating [2.2] paracyclophane in a partial vacuum gives rise to a diradical species, which polymerizes when deposited on a surface. Until the “monomer” comes into contact with a surface it is in a gaseous phase and can access the entire exposed surface. A biomedical device with a parylene coating can then be further treated to produce polar reactive groups at the surface, so that antimicrobial peptides can chemically react with the modified parylene coated surface. Methods to produce polar reactive groups on plastic and metallic materials are known in the art, and may include for example ozone treatments, corona discharge, flame treatments, plasma processing, acid etching, ultraviolet light irradiation, gamma ray irradiation, and electron bean1 irradiation. It is recognized that these surface modification techniques can be used on most polymeric surfaces to produce reactive groups to which the antimicrobial peptides can form covalent bonds and become immobilized on the dental medical device surface.

Entrapment of antimicrobial peptides in a polymer matrix that is applied on a medical device surface is another means of preparing an antimicrobial coating in embodiments of the present invention. Application of non-reactive polymer matrices is used for indwelling medical devices. Polymer matrices with antimicrobial peptides could be used to construct a device or applied as a coating on a device. In embodiments, the antimicrobial peptides may not be chemically reacted with the polymer matrix. The peptides may then slowly diffuse out of the matrix and work as a bactericide in the vicinity of the surface. A sustained release of the antimicrobial peptides is possible, resulting in longer term protection from infection. An exemplary, but not limiting, polymer matrix is one of polyurethane.

In useful embodiments of the present invention it may be desirable for antimicrobial peptides to both be covalently bonded to the surface of a dental medical device, and to exhibit a sustained release or elution from the surface of a medical device. In such scenarios, the total of the eluting phase and the covalently bonded phase provides protection and/or treatment in the first instances after insertion of the medical device. As the eluting phase is depleted the covalently bonded phase may provide long term protection or protection from future infections.

For example, an antimicrobial peptide may be provided to the treated surface of a dental device in excess of what can covalently bond with the surface. The excess may be entrapped in a polymeric matrix of the surface treatment, or alternatively, or in addition, otherwise bonded through hydrogen bonding or dispersion forces between the peptides, for example.

Other means of treating surfaces or depositing coatings on surfaces such as plasma vapor deposition (PVD), chemical vapor deposition (CVD), graft polymerization are also techniques that can be used to immobilize antimicrobial peptides on a medical device surface. Any surface modification technique that is known now or hereafter to one or ordinary skill in the art, which can be used to enable covalent bonding or other method of immobilization of antimicrobial peptides on a medical device surface, or to facilitate elution from a medical device surface are within the scope of this invention.

The antimicrobial peptides of the present invention are peptides which exhibit antimicrobial activity against diverse microorganisms. Preferably the antimicrobial peptides are alpha-helical, more preferably the antimicrobial peptides are selected from groups designated herein as LLPs, LBUs, and eCAPs. The LLPs, LBUs, and eCAPs classes of peptides are further disclosed and described below. In one aspect of the invention, the peptides correspond to amino acid sequences in the transmembrane (TM) proteins of lentiviruses, in particular, HIV and SIV. These peptides comprise arginine-rich sequences, which, when modeled for secondary structure, display high amphipathicity and hydrophobic moment. The antimicrobial peptides are highly inhibitory to microorganisms but significantly less toxic to red blood cells and other normal mammalian cells. As a result, these peptides can be characterized as selective antimicrobial agents.

In a useful embodiment of the present invention, the antimicrobial peptides may retain antibacterial activity after a dental device coated with the antimicrobial, peptide is sterilized. It is reported below that the WLBU-2 peptide coated on a substrate retained antimicrobial activity after ethylene oxide sterilization. Preliminary experiments on neat solid antimicrobial peptides disclosed herein or liquid solutions of peptides disclosed herein have demonstrated that several of the peptides remained biologically active after low and high gamma-irradiation, and high temperature sterilization processes.

This invention and embodiments illustrating the method and materials used may be further understood by reference to the following non-limiting examples.

### Example 1

Medical standard alloy 316L stainless steel pins were coated with an antimicrobial peptide layer to determine the efficacy of an immobilized antimicrobial peptide. The pins were coated with a tie layer. A parylene coating containing a reactive molecule specific for the N-terminus region of the peptide (“Photopolymer”) was then applied over the tie layer. The peptide WLBU-2 (SEQ ID NO. 179) was applied to the parylene coating and exposed to UV light to facilitate covalent bonding of the peptide with the parylene coating. Parylene coated stainless steel pins and non-coated stainless steel pins were used as controls. All samples and controls were subjected to ethylene oxide sterilization. The coated samples were placed in 75% relative humidity overnight. The immobilized antimicrobial peptide was tested in an in-vitro assay system showing attachment of the antimicrobial peptide to the metal implant and efficacy against Staphylococcus aureus (data not shown).

### Example 2

Medical standard plastic endodontic obturators were coated with an antimicrobial peptide layer to determine the efficacy of an immobilized antimicrobial peptide. The obturators had a layer of gutta-percha over ⅔ the length of the shaft. The obturators were coated with a tie layer. A parylene coating containing a reactive molecule specific for the N-terminus region of the peptide (“Photopolymer”) was then applied over the tie layer. The peptide WLBU-2 (SEQ ID NO. 179) was applied to the parylene coating and exposed to UV light to facilitate covalent bonding of the peptide with the parylene coating. Non-coated obturators were used as controls. All samples and controls were subjected to ethylene oxide sterilization. The coated samples were placed in 75% relative humidity overnight.

### Example 3

The obturators of Example 2 were exposed to Staphylococcus aureus(University of Calgary clinical isolate, strain 2654) using an in-vitro assay system outlined below.

An in-vitro assay system for evaluating adhesion, post adhesion killing and inhibition of biofilm formation on surfaces is outlined below. Using a cryogenic stock (−70° C.), a first sub-culture was streaked out of the bacterial organisms listed above on TSA. The plate was incubated at 37±1° C. for 24 hours in 5.0% CO2 and the plate was stored wrapped in parafilm at 4° C. From the first sub-culture, a second sub-culture was streaked out on TSA and incubated at 37±1° C. for 24 hours in 5.0% CO2. The second sub-culture was used within 24 hours starting from the time it was first removed from incubation. Using the second sub-culture an inoculum was created in 3 ml sterile water that matched a 0.5 McFarland standard (1.5×10^{8th} cells per ml) in a glass test tube using a sterile cotton swab. The inoculum was adjusted to an approximate cell density of 10⁶ CFU/ml in 10% Mueller Hinton broth in PBS and 20 ml of the inoculum was placed in the wells of the EO-BEST Device. A growth control with no stainless steel was made as well. The lid with the attached samples was inserted into the inoculum and the entire device was incubated at 37±1° C. for 24 hours in 5.0% CO2. After the 24 hours, the samples on the lid are rinsed three times in sterile saline by dipping the lid with samples attached into three consecutive bottom plates containing 23 ml of sterile saline in each well. The lid with samples attached was placed into wells containing 23 ml of sterile saline for 2 hours to allow recently adhered bacteria to be acted upon by any antimicrobial in the coating. The EO-BEST lid was then inserted into wells containing 23 ml of sodium thioglycollate (0.1% w/v) and 0.5% Tween 80 in phosphate buffered saline (PBS) in each well. The entire device assembled above was sonicated for 30 minutes. Following sonication, 100 ml from each well of the EO-BEST plate was placed into the first 12 empty wells of the first row of a 96 well-micro titer plate. 180 ml of 0.9% sterile saline was placed in the remaining rows. A serial dilution (10°±10⁵) was prepared by moving 20 ml down each of the 8 rows. 20 ml was removed from each well and spot plated on a prepared Trypticase Soy Agar plate. Plates were incubated at 37±1° C. in 5.0% CO2 and counted after approximately 24 hours of incubation.

CFU total (per vial) of Enterococcus faecalis (ME 46789) on coated and uncoated (control) obturators is found the following Table 5.

TABLE 5

| | | Internal Replicate | | |
|---|---|---|---|---|
| | | (LOG 10 CFU) | | |
| Sample | Sample Description | 1 | 2 | 3 |
| A | Control | 4.65 | 4.70 | 4.74 |
| B | Peptide Coated | 0.00 | 0.00 | 0.00 |

There was significantly reduced growth (no bio-film growth at all) of Enterococcus Faecalis ME 46789 on the peptide coated obturators compared to the control, uncoated obturators, under the test conditions of this experiment.

The peptide coated obturators were effective in preventing biofilm adherence, and preventing biofilm formation compared to the uncoated obturators, under the test conditions of this experiment.

Although the present invention has been described in considerable detail with reference to certain preferred embodiments thereof, other versions are possible. Therefore the spirit and scope of the appended claims should not be limited to the description and the preferred versions contained within this specification.

1. A dental implant comprising:
a biologically compatible surface, wherein at least a portion of the surface further includes an antimicrobial peptide selected from the group consisting of a linear lytic peptide, a lytic base unit peptide, and an engineered cationic antimicrobial peptide.

2-4. (canceled)

5. The dental implant of claim 1, wherein the antimicrobial peptide is immobilized.

6. The dental implant of claim 1, wherein substantially all of the surface is covered by a therapeutically effective amount of the antimicrobial peptide.

7. The dental implant of claim 1 further comprising a linking layer between the biologically compatible surface and the antimicrobial peptide.

8. The dental implant of claim 1, wherein the dental implant is an obturator.

9. A method for immobilizing antimicrobial peptides on a dental implant, comprising:
providing a dental implant;
applying a linking layer to at least a portion of a surface of the dental implant, wherein the linking layer is applied by a methodology selected from the group consisting of silanization, polyxylylene polymer coating, plasma vapor deposition, chemical vapor deposition, surfactant coating, photochemical reactive coating, and block copolymer coating; and providing an antimicrobial peptide on said dental implant, said antimicrobial peptide selected from a group consisting of a linear lytic peptide, a lytic base unit peptide and an engineered cationic antimicrobial peptide to the linking layer.

10-12. (canceled)

13. The method of claim 9, wherein a method of applying a linking layer is selected from the group consisting of silanization, polyxylylene polymer coating, plasma vapor deposition, chemical vapor deposition, surfactant coating, photochemical reactive coating, and block copolymer coating.

14. An endodontic instrument, comprising: wherein the endodontic instrument comprises an elongated shaft and handle portion, wherein the elongated shaft is covered with at least one layer comprising a filling material and an antimicrobial peptide selected from a linear lytic peptide, a lytic base unit peptide and an engineered cationic antimicrobial peptide.

15-18. (canceled)

19. The endodontic instrument of claim 14, wherein the filling material is in contact with the elongated shaft and the antimicrobial peptide comprises a layer over the filling material.

20. The endodontic instrument of claim 14, wherein the antimicrobial peptide is in contact with the elongated shaft and the filling material comprises a layer over the filling material.

21. The endodontic instrument of claim 14, wherein the antimicrobial peptide and the filling material comprise a substantially uniformly mixed composite layer in contact with the elongated shaft.

22. The endodontic instrument of claim 14, wherein the antimicrobial peptide covers substantially the entire length of the elongated shaft.

23-37. (canceled)

38. The dental implant of claim 1, wherein the linear lytic peptide is selected from the group consisting of SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, SEQ. ID NO. 18, SEQ. ID NO. 19, SEQ. ID NO. 20, SEQ. ID NO. 21, SEQ. ID NO. 22, SEQ. ID NO. 23, SEQ. ID NO. 24, SEQ. ID NO. 25, SEQ. ID NO. 26, SEQ. ID NO. 27, SEQ. ID NO. 28, SEQ. ID NO. 29, SEQ. ID NO. 30, SEQ. ID NO. 31, SEQ. ID NO. 32, SEQ. ID NO. 33, SEQ. ID NO. 34, SEQ. ID NO. 35, SEQ. ID NO. 36, SEQ. ID NO. 37, SEQ. ID NO. 38, SEQ. ID NO. 39, SEQ. ID NO. 40, SEQ. ID NO. 41, SEQ. ID NO. 42, SEQ. ID NO. 43, SEQ. ID NO. 44, SEQ. ID NO. 45, SEQ. ID NO. 46, SEQ. ID NO. 47, SEQ. ID NO. 48, SEQ. ID NO. 49, SEQ. ID NO. 50, SEQ. ID NO. 51, SEQ. ID NO. 52, SEQ. ID NO. 53, SEQ. ID NO. 54, SEQ. ID NO. 55, SEQ. ID NO. 56, SEQ. ID NO. 57, SEQ. ID NO. 58, SEQ. ID NO. 59, SEQ. ID NO. 60, SEQ. ID NO. 61, SEQ. ID NO. 62, SEQ. ID NO. 63, SEQ. ID NO. 64, SEQ. ID NO. 65, SEQ. ID NO. 66, SEQ. ID NO. 67, SEQ. ID NO. 68, SEQ. ID NO. 69, SEQ. ID NO. 70, SEQ. ID NO. 71, SEQ. ID NO. 72, SEQ. ID NO. 73, SEQ. ID NO. 74, SEQ. ID NO. 75, SEQ. ID NO. 76, SEQ. ID NO. 77, SEQ. ID NO. 78, SEQ. ID NO. 79, SEQ. ID NO. 80, SEQ. ID NO. 81, SEQ. ID NO. 82, SEQ. ID NO. 83, SEQ. ID NO. 84, SEQ. ID NO. 85, SEQ. ID NO. 86, SEQ. ID NO. 87, SEQ. ID NO. 88, SEQ. ID NO. 89, SEQ. ID NO. 90, SEQ. ID NO. 91, SEQ. ID NO. 92, SEQ. ID NO. 93, SEQ. ID NO. 94, SEQ. ID NO. 95, SEQ. ID NO. 96, SEQ. ID NO. 97, SEQ. ID NO. 98, SEQ. ID NO. 99, SEQ. ID NO. 100, SEQ. ID NO. 101, SEQ. ID NO. 102, SEQ. ID NO. 103, SEQ. ID NO. 104, SEQ. ID NO. 105, SEQ. ID NO. 106, SEQ. ID NO. 107, SEQ. ID NO. 108, SEQ. ID NO. 109, SEQ. ID NO. 110, SEQ. ID NO. 111, SEQ. ID NO. 112, SEQ. ID NO. 113, SEQ. ID NO. 114, SEQ. ID NO. 115, SEQ. ID NO. 116, SEQ. ID NO. 117, SEQ. ID NO. 118, SEQ. ID NO. 119, SEQ. ID NO. 120, SEQ. ID NO. 121, SEQ. ID NO. 122, SEQ. ID NO. 123, SEQ. ID NO. 124, SEQ. ID NO. 125, SEQ. ID NO. 126, SEQ. ID NO. 127, SEQ. ID NO. 128, SEQ. ID NO. 129, SEQ. ID NO. 130, SEQ. ID NO. 131, SEQ. ID NO. 132, SEQ. ID NO. 133, SEQ. ID NO. 134, SEQ. ID NO. 135, SEQ. ID NO. 136, SEQ. ID NO. 137, SEQ. ID NO. 138, SEQ. ID NO. 139, SEQ. ID NO. 140, SEQ. ID NO. 141, SEQ. ID NO. 142, SEQ. ID NO. 143, SEQ. ID NO. 144, SEQ. ID NO. 145, SEQ. ID NO. 146, SEQ. ID NO. 147, SEQ. ID NO. 148, SEQ. ID NO. 149, SEQ. ID NO. 150, SEQ. ID NO. 151, SEQ. ID NO. 152, SEQ. ID NO. 153, SEQ. ID NO. 154, SEQ. ID NO. 155, SEQ. ID NO. 156, SEQ. ID NO. 157, SEQ. ID NO. 158, SEQ. ID NO. 159, SEQ. ID NO. 160, SEQ. ID NO. 161, SEQ. ID NO. 162, SEQ. ID NO. 163, SEQ. ID NO. 164, SEQ. ID NO. 165, SEQ. ID NO. 166, SEQ. ID NO. 167, SEQ. ID NO. 168 and SEQ. ID NO. 169.

39. The dental implant of claim 1, wherein the linear lytic peptide is selected from the group consisting of SEQ ID NO. 4; SEQ ID NO. 5, SEQ ID NO. 8, SEQ ID NO. 9, SEQ ID NO. 10, SEQ ID NO. 38, SEQ ID NO. 73, SEQ ID NO. 92, and SEQ ID NO. 97.

40. The dental implant of claim 1, wherein the lytic base unit peptide is selected from the group consisting of SEQ. ID NO. 170, SEQ. ID NO. 171, SEQ. ID NO. 172, SEQ. ID NO. 173, SEQ. ID NO. 174, SEQ. ID NO. 175, SEQ. ID NO. 176, SEQ. ID NO. 177, SEQ. ID NO. 178, SEQ. ID NO. 179, SEQ. ID NO. 180 and SEQ. ID NO. 181.

41. The method of claim 9, wherein the linear lytic peptide is selected from SEQ. ID NO. 1, SEQ. ID NO. 2, SEQ. ID NO. 3, SEQ. ID NO. 4, SEQ. ID NO. 5.

42. The method of claim 9, wherein the linear lytic peptide is selected from SEQ ID NO. 4, SEQ ID NO. 5, SEQ ID NO. 8, SEQ ID NO. 9, SEQ ID NO. 10, SEQ ID NO. 38, SEQ ID NO. 73, SEQ ID NO. 92, and SEQ ID NO. 97.

43. The method of claim 9, wherein the lytic base unit peptide is selected from SEQ. ID NO. 170, SEQ. ID NO. 171, SEQ. ID NO. 172, SEQ. ID NO. 173, SEQ. ID NO. 174, SEQ. ID NO. 175, SEQ. ID NO. 176, SEQ. ID NO. 177, SEQ. ID NO. 178, SEQ. ID NO. 179, SEQ. ID NO. 180 and SEQ. ID NO. 181.

44. The endodontic instrument of claim 14, wherein the linear lytic peptide is selected from the group consisting of SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, SEQ. ID NO. 18, SEQ. ID NO. 19, SEQ. ID NO. 20, SEQ. ID NO. 21, SEQ. ID NO. 22, SEQ. ID NO. 23, SEQ. ID NO. 24, SEQ. ID NO. 25, SEQ. ID NO. 26, SEQ. ID NO. 27, SEQ. ID NO. 28, SEQ. ID NO. 29, SEQ. ID NO. 30, SEQ. ID NO. 31, SEQ. ID NO. 32, SEQ. ID NO. 33, SEQ. ID NO. 34, SEQ. ID NO. 35, SEQ. ID NO. 36, SEQ. ID NO. 37, SEQ. ID NO. 38, SEQ. ID NO. 39, SEQ. ID NO. 40, SEQ. ID NO. 41, SEQ. ID NO. 42, SEQ. ID NO. 43, SEQ. ID NO. 44, SEQ. ID NO. 45, SEQ. ID NO. 46, SEQ. ID NO. 47, SEQ. ID NO. 48, SEQ. ID NO. 49, SEQ. ID NO. 50, SEQ. ID NO. 51, SEQ. ID NO. 52, SEQ. ID NO. 53, SEQ. ID NO. 54, SEQ. ID NO. 55, SEQ. ID NO. 56, SEQ. ID NO. 57, SEQ. ID NO. 58, SEQ. ID NO. 59, SEQ. ID NO. 60, SEQ. ID NO. 61, SEQ. ID NO. 62, SEQ. ID NO. 63, SEQ. ID NO. 64, SEQ. ID NO. 65, SEQ. ID NO. 66, SEQ. ID NO. 67, SEQ. ID NO. 68, SEQ. ID NO. 69, SEQ. ID NO. 70, SEQ. ID NO. 71, SEQ. ID NO. 72, SEQ. ID NO. 73, SEQ. ID NO. 74, SEQ. ID NO. 75, SEQ. ID NO. 76, SEQ. ID NO. 77, SEQ. ID NO. 78, SEQ. ID NO. 79, SEQ. ID NO. 80, SEQ. ID NO. 81, SEQ. ID NO. 82, SEQ. ID NO. 83, SEQ. ID NO. 84, SEQ. ID NO. 85, SEQ. ID NO. 86, SEQ. ID NO. 87, SEQ. ID NO. 88, SEQ. ID NO. 89, SEQ. ID NO. 90, SEQ. ID NO. 91, SEQ. ID NO. 92, SEQ. ID NO. 93, SEQ. ID NO. 94, SEQ. ID NO. 95, SEQ. ID NO. 96, SEQ. ID NO. 97, SEQ. ID NO. 98, SEQ. ID NO. 99, SEQ. ID NO. 100, SEQ. ID NO. 101, SEQ. ID NO. 102, SEQ. ID NO. 103, SEQ. ID NO. 104, SEQ. ID NO. 105, SEQ. ID NO. 106, SEQ. ID NO. 107, SEQ. ID NO. 108, SEQ. ID NO. 109, SEQ. ID NO. 110, SEQ. ID NO. 111, SEQ. ID NO. 112, SEQ. ID NO. 113, SEQ. ID NO. 114, SEQ. ID NO. 115, SEQ. ID NO. 116, SEQ. ID NO. 117, SEQ. ID NO. 118, SEQ. ID NO. 119, SEQ. ID NO. 120, SEQ. ID NO. 121, SEQ. ID NO. 122, SEQ. ID NO. 123, SEQ. ID NO. 124, SEQ. ID NO. 125, SEQ. ID NO. 126, SEQ. ID NO. 127, SEQ. ID NO. 128, SEQ. ID NO. 129, SEQ. ID NO. 130, SEQ. ID NO. 131, SEQ. ID NO. 132, SEQ. ID NO. 133, SEQ. ID NO. 134, SEQ. ID NO. 135, SEQ. ID NO. 136, SEQ. ID NO. 137, SEQ. ID NO. 138, SEQ. ID NO. 139, SEQ. ID NO. 140, SEQ. ID NO. 141, SEQ. ID NO. 142, SEQ. ID NO. 143, SEQ. ID NO. 144, SEQ. ID NO. 145, SEQ. ID NO. 146, SEQ. ID NO. 147, SEQ. ID NO. 148, SEQ. ID NO. 149, SEQ. ID NO. 150, SEQ. ID NO. 151, SEQ. ID NO. 152, SEQ. ID NO. 153, SEQ. ID NO. 154, SEQ. ID NO. 155, SEQ. ID NO. 156, SEQ. ID NO. 157, SEQ. ID NO. 158, SEQ. ID NO. 159, SEQ. ID NO. 160, SEQ. ID NO. 161, SEQ. ID NO. 162, SEQ. ID NO. 163, SEQ. ID NO. 164, SEQ. ID NO. 165, SEQ. ID NO. 166, SEQ. ID NO. 167, SEQ. ID NO. 168 and SEQ. ID NO. 169.

45. The endodontic instrument of claim 14, wherein the engineered cationic antimicrobial peptide is selected from SEQ. ID NO. 182, SEQ ID NO. 183, SEQ. ID NO. 184, and SEQ. ID NO. 185.
